(19) United States
(12) Patent Application Publication
(10) Pub. No.: US 2009/0257999 A1
(43) Pub. Date: Oct. 15, 2009

(54) Method of preventing contrast-induced nephropathy

(75) Inventor: Mitchell P. Fink, Beverly, MA (US)

Correspondence Address: LAHIVE & COCKFIELD, LLP;FLOOR 30, SUITE 3000, ONE POST OFFICE SQUARE, BOSTON, MA, 02109, US

(73) Assignee: Inotek Pharmaceuticals Corporation, Beverly, MA (US)

(21) Appl. No.: 12/317,922

(22) Filed: Dec. 30, 2008

Related U.S. Application Data
(60) Provisional application No. 61/009,600, filed on Dec. 31, 2007.

(51) Int. Cl. A61K 38/44; A61K 31/444; A61K 31/409; A61K 31/555

(52) U.S. Cl. 424/94.4; 514/184

(57) ABSTRACT

The present invention relates to methods of preventing contrast-induced nephropathy including the step of administering an effective amount of a compound (e.g., a peroxynitrite decomposition agent, a PARP inhibitor or a superoxide dismutase mimic) to a subject to be administered a contrast agent.

### RELATED APPLICATIONS

This application claims priority to U.S. Provisional Patent Application No. 61/009,600, filed on Dec. 31, 2007, the entire contents of which are hereby incorporated by reference.

### BACKGROUND OF THE INVENTION

Contrast-induced nephropathy (CIN) is generally recognized as acute renal failure occurring within 48 hours of exposure to intravascular contrast material, and where other causes of renal failure are not attributable. Its presence is generally determined when an increase in serum creatinine levels is exhibited in a subject who has been exposed to intravascular contrast material. Contrast-induced morbidity has become a significant cause of hospital morbidity and mortality with the increasing use of iodinated contrast media in diagnostic imaging and interventional procedures such as angiography. In 2003, over 80 million doses of iodinated intravascular contrast media were administered, corresponding to approximately 8 million liters according to Katzberg et. al. Kidney International (2006) 69, S3-S7. There are different classes of contrast agents in use such as:
High osmolar agents, such as Iothalamate and Diatrizoate; where the osmolality of these agents is about 5 times greater than the osmolality of blood;
Low osmolar agents, such as Iohexyl, ioversol, Iopamidol, iopromide, Iomeprol and Ioxaglate; where the osmolality of these agents is about 2-3 times greater than the osmolality of blood; and

Iso-osmolar agents such as Iotrolan and Iodixanol; where the osmolality of these agents is the same as the osmolality of blood.

The pathophysiological mechanisms that underly the development of CIN are not fully understood (Persson et. al. Kidney International (2006) 69, S8-S10). Nevertheless, there are recognized risk factors that pre-dispose individuals for the development of contrast agent-induced acute renal failure and these include subject-related factors and procedure-related factors as shown in the Table from Heyman et al Diseases of the Kidney and Urinary Tract, Eighth Edition, Volume III, Chapter 45 pages 1099-1120, page 1101.

| Patient related Factors | Procedure related Factors |
|---|---|
| Renal insufficiency | Types of radiocontrast medium |
| | (High osmolar > low or isoosmolar) |
| Diabetes mellitus | Dose of radiocontrast medium |
| Age | Repeated exposures to radiocontrast |
| | material within 72 hr |
| Effective volume depletion | Mode of administration |

| | |
|---|---|
| Dehydration | (Intraarterial > intravenous) |
| Congestive heart failure | |
| Chronic heart failure | |
| Chronic liver disease | |
| Nephrotic syndrome | |
| Concomitant hypotension | |

Concomitant exposure to
nephrotoxins

Medications
Other exogenous nephrotoxins
Sepsis

Myeloma
Male gender
Hypertension
Transplanted kidney
Hyperuricemia
Proteinuria
Anemia

There is a clear need for compounds, compositions and methods that are useful for treating or preventing contrast-induced nephropathy.

### SUMMARY OF THE INVENTION

The present invention provides a method of preventing contrast-induced nephropathy including the step of administering an effective amount of a peroxynitrite decomposition agent to a subject to be administered a contrast agent.

The present invention also provides a peroxynitrite decomposition agent for use in the prevention of contrast-induced nephropathy in a subject to be administered a contrast agent.

In one embodiment, the peroxynitrite decomposition agent is administered to the subject prior to administration of a contrast agent.

In another embodiment, the peroxynitrite decomposition agent is administered to the subject simultaneously with the administration of the contrast agent.

In another embodiment, the peroxynitrite decomposition agent is administered to the subject after the administration of the contrast agent.

In one embodiment, the contrast agent is selected from Iothalamate, Metrizoate, Diatrizoate, Ioxilan Iohexyl, Ioversol, Iopamidol, Iopromide, Iomeprol, Ioxaglate, Iotrolan and Iodixanol.

In another embodiment, the contrast agent is selected from Iomeprol.

In another embodiment, the peroxynitrite decomposition agent is administered to the subject in an amount of between 1 ng/kg to 1000 mg/kg. In a further embodiment, the peroxynitrite decomposition agent is administered to the subject in an amount of between 0.01 mg to 100 mg.

In another embodiment, the peroxynitrite decomposition agent is administered to the subject in an amount of between 0.1 mg/kg to 10 mg/kg.

In another embodiment, the peroxynitrite decomposition agent is administered to the subject in an amount of between 0.1 mg/kg to 1 mg/kg.

In one embodiment, the peroxynitrite decomposition agent is a metalloporphyrin selected from a compound having the formula

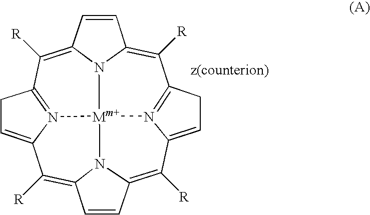

wherein:

M is Fe or Mn;

m is 0 or 1;

each R is independently selected from

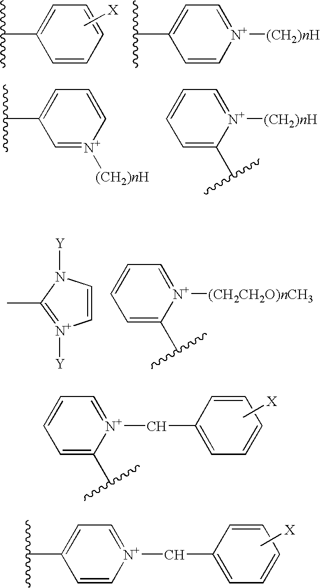

where X is selected from halogen, alkyl, —C(O)OH, —C(O)O⁻ or —C(O)(amino acid residue), SO₂OH, SO₂O— or SO₂(amino acid residue);

where each Y is independently selected from halogen, C₁-C₆ alkyl, C₁-C₆ alkyl-O—C₁-C₆alkyl,

each n is independently an integer from 1 to 4.

Z is the number of counterions sufficient to balance the charges of the compound of Formula (A).

In a further embodiment, wherein X is —C(O)(amino acid residue), the amino acid of the amino acid residue is β-alanine, γ-aminobutyric acid, 6-aminohexanoic acid, 5-aminovaleric acid, L-aspartic acid, L-glutamine, L-glutamic acid, glycine, L-phenylalanine, L-tyrosine, or L-valine.

In one embodiment, the counterion is Cl⁻ or Br⁻.

In one aspect, the metalloporphyrin is selected from a compound having the formula

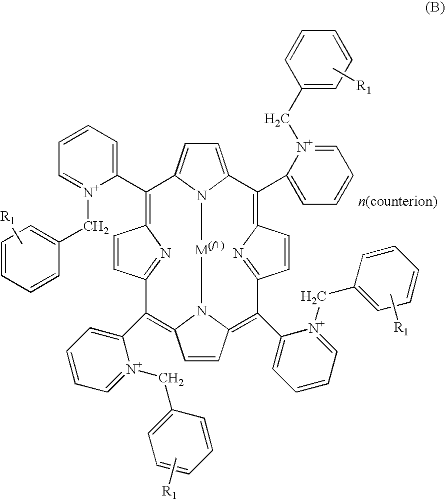

wherein:

M is Fe or Mn;

f is 0 or 1;

each R₁ is independently —C(O)OH, —C(O)O⁻ or —C(O)(amino acid residue) or SO₂(amino acid residue); and

n is the number of counterions sufficient to balance the charges of the compound of Formula (B).

In one embodiment the counterion is Cl⁻ or Br⁻.

In one embodiment, the amino acid of the amino acid residue is β-alanine, γ-aminobutyric acid, 6-aminohexanoic acid, 5-aminovaleric acid, L-aspartic acid, L-glutamine, L-glutamic acid, glycine, L-phenylalanine, L-tyrosine, or L-valine.

In one aspect, the amino acid residue is L-tyrosine.

In one embodiment, the metalloporphyrin is selected from

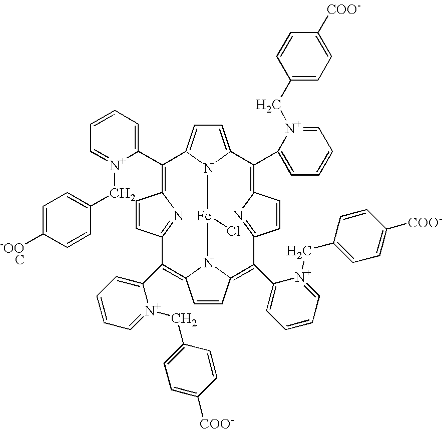

In another embodiment, the peroxynitrite decomposition agent is administered in combination with one or more of the following selection:

Prostaglandin, an adenosine antagonist, E₁; N-acetylcysteine (NAC), sodium bicarbonate, a calcium channel blocker, ascorbic acid, misoprostol, an ACE inhibitor, deferiprone, a PARP inhibitor, a superoxide dismutase (SOD) mimic, alpha-phenyl-N-tert-butyl nitrone, 2,4-disulphonyl-N-tert-butyl nitrone, 2-sulphonyl-N-tert-butyl nitrone, EPO and melatonin.

The present invention further provides a method of preventing contrast-induced nephropathy including the step of administering an effective amount of a superoxide dismutase mimic to a subject to be administered a contrast agent.

The present invention also provides a superoxide dismutase mimic for use in the prevention of contrast-induced nephropathy in a subject to be administered a contrast agent.

In one embodiment, the superoxide dismutase mimic is administered to the subject prior to administration of a contrast agent.

In another embodiment, the superoxide dismutase mimic is administered to the subject simultaneously with the administration of the contrast agent.

In another embodiment, the superoxide dismutase mimic is administered to the subject after the administration of the contrast agent.

In one embodiment, the contrast agent is selected from as Iothalamate, Metrizoate, Diatrizoate, Ioxilan Iohexyl, Ioversol, Iopamidol, Iopromide, Iomeprol, Ioxaglate, Iotrolan and Iodixanol.

In another embodiment, the contrast agent is selected from Iomeprol.

In another embodiment, the superoxide dismutase mimic is administered to the subject in an amount of between 1 ng/kg to 1000 mg/kg. In a further embodiment, the peroxynitrite decomposition agent is administered to the subject in an amount of between 0.01 mg/kg to 100 mg/kg.

In another embodiment, the superoxide dismutase mimic is administered to the subject in an amount of between 0.1 mg/kg to 10 mg/kg.

In another embodiment, the superoxide dismutase mimic is administered to the subject in an amount of between 0.1 mg/kg to 1 mg/kg.

In another embodiment, the superoxide dismutase mimic is selected from manganese tetrakis (4-benzoic acid) porphyrin, M40403, M40419 and AEOL 10113.

In another embodiment, the superoxide dismutase mimic is administered in combination with a peroxynitrite decomposition agent, N-acetylcysteine (NAC), sodium bicarbonate, a calcium channel blocker, ascorbic acid, prostaglandin E₁, misoprostol, an ACE inhibitor, deferiprone, a PARP inhibitor, alpha-phenyl-N-tert-butyl nitrone, 2,4-disulphonyl-N-tert-butyl nitrone, 2-sulphonyl-N-tert-butyl nitrone, a superoxide dismutase mimetic, EPO, an adenosine antagonist or melatonin.

In one embodiment, the superoxide dismutase mimic is administered in combination with a peroxynitrite decomposition metalloporphyrin selected from a compound having the formula

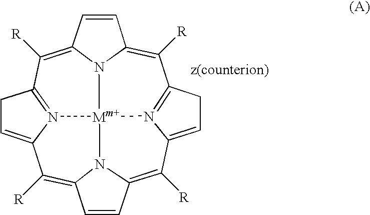

wherein:

M is Fe or Mn;

m is 0 or 1;

each R is independently selected from

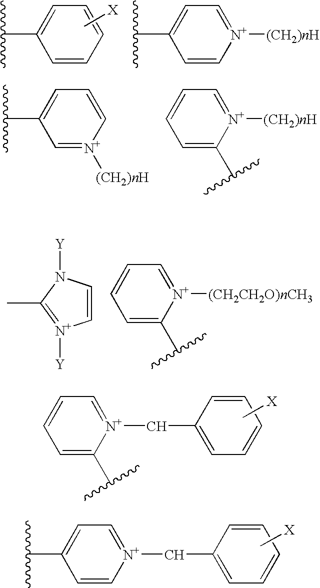

where X is selected from halogen, alkyl, —C(O)OH, —C(O)O⁻ or —C(O)(amino acid residue), SO₂OH, SO₂O⁻ or SO₂(amino acid residue);

where each Y is independently selected from halogen, C₁-C₆ alkyl, C₁-C₆ alkyl-O—C₁-C₆alkyl,

each n is independently an integer from 1 to 4,

Z is the number of counterions sufficient to balance the charges of the compound of Formula (A).

In a further embodiment, wherein X is —C(O)(amino acid residue), the amino acid of the amino acid residue is β-alanine, γ-aminobutyric acid, 6-aminohexanoic acid, 5-aminovaleric acid, L-aspartic acid, L-glutamine, L-glutamic acid, glycine, L-phenylalanine, L-tyrosine, or L-valine.

In one embodiment, the counterion is Cl⁻ or Br⁻.

In one aspect, the metalloporphyrin is selected from a compound having the formula

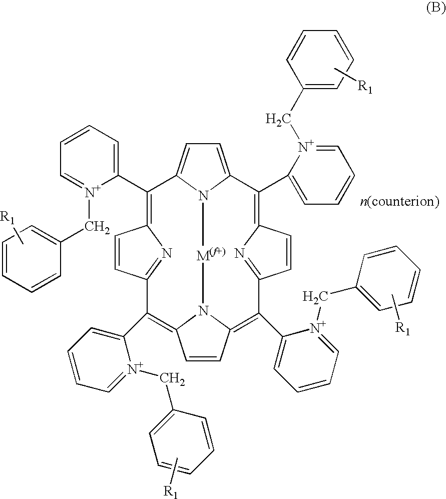

wherein:

M is Fe or Mn;

f is 0 or 1;

each R₁ is independently —C(O)OH, —C(O)O⁻ or —C(O)(amino acid residue) or SO₂(amino acid residue); and

n is the number of counterions sufficient to balance the charges of the compound of Formula (B).

In one embodiment, the counterion is Cl⁻ or Br⁻.

In one embodiment, the amino acid of the amino acid residue is β-alanine, γ-aminobutyric acid, 6-aminohexanoic acid, 5-aminovaleric acid, L-aspartic acid, L-glutamine, L-glutamic acid, glycine, L-phenylalanine, L-tyrosine, or L-valine.

In one aspect, the amino acid residue is L-tyrosine.

In one embodiment, the metalloporphyrin is selected from

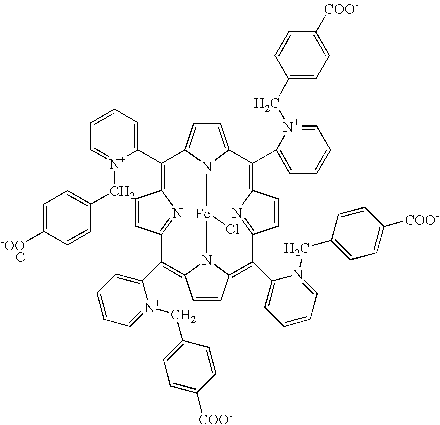

The present invention further provides a method of preventing contrast-induced nephropathy including the step of administering an effective amount of a PARP inhibitor to a subject to be administered a contrast agent.

In one embodiment, the PARP inhibitor is administered to the subject prior to administration of a contrast agent.

In another embodiment, the PARP inhibitor is administered to the subject simultaneously with the administration of the contrast agent.

In a further embodiment, the PARP inhibitor is administered to the subject after the administration of the contrast agent.

In the above embodiments, the contrast agent is selected from as Iothalamate, Metrizoate, Diatrizoate, Ioxilan Iohexyl, Ioversol, Iopamidol, Iopromide, Iomeprol, Ioxaglate, Iotrolan and Iodixanol.

In one aspect, the contrast agent is selected from Iomeprol.

In one embodiment, the PARP inhibitor is administered to the subject in an amount of between 1 ng/kg to 1000 mg/kg.

In one embodiment, the PARP inhibitor is administered to the subject in an amount of between 0.01 mg/kg to 100 mg/kg.

In one embodiment, the PARP inhibitor is administered to the subject in an amount of between 0.1 mg/kg to 10 mg/kg.

In one embodiment, the PARP inhibitor is administered to the subject in an amount of between 0.1 mg/kg to 1 mg/kg.

In one embodiment, the PARP inhibitor is selected from INO 1001, PJ34, ABT888, AG14699, AG14361, KU59346, BSI 201 and GPI 21016.

In another embodiment, the PARP inhibitor is administered in combination with a peroxynitrite decomposition agent, N-acetylcysteine (NAC), sodium bicarbonate, a calcium channel blocker, ascorbic acid, prostaglandin E₁, misoprostol, an ACE inhibitor, deferiprone, alpha-phenyl-N-tert-butyl nitrone, 2,4-disulphonyl-N-tert-butyl nitrone, 2-sulphonyl-N-tert-butyl nitrone, a superoxide dismutase mimetic, an adenosine antagonist, EPO or melatonin.

In one embodiment, the PARP inhibitor is administered in combination with N-acetylcysteine (NAC).

In another embodiment, the PARP inhibitor is administered in combination with a peroxynitrite decomposition agent.

In one embodiment, the peroxynitrite decomposition agent is a metalloporphyrin selected from a compound having the formula

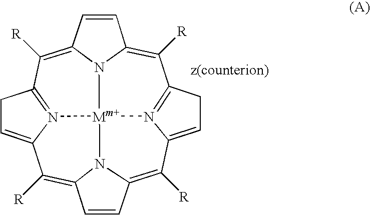

wherein:

M is Fe or Mn;

m is 0 or 1;

each R is independently selected from

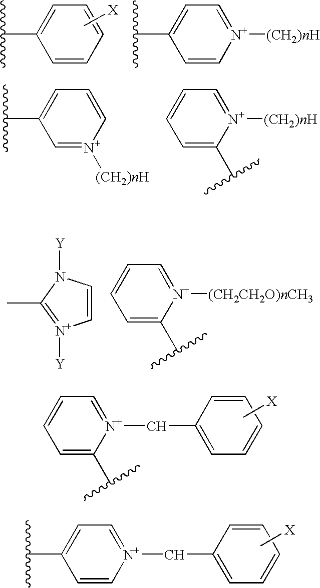

where X is selected from halogen, alkyl, —C(O)OH, —C(O)O⁻ or —C(O)(amino acid residue), SO₂OH, SO₂O⁻ or SO₂(amino acid residue);

where each Y is independently selected from halogen, C₁-C₆ alkyl, C₁-C₆ alkyl-O—C₁-C₆alkyl,

each n is independently an integer from 1 to 4.

Z is the number of counterions sufficient to balance the charges of the compound of Formula (A).

In another embodiment, the metalloporphyrin is selected from a compound having the formula

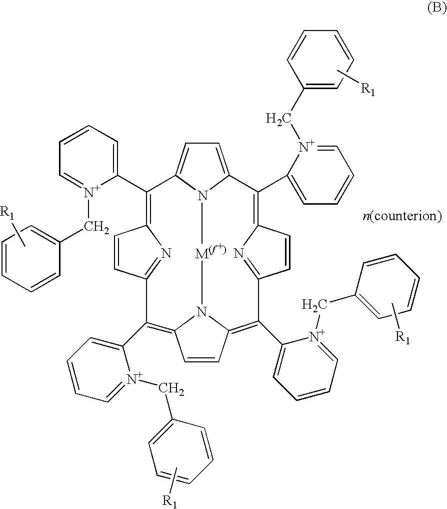

wherein:

M is Fe or Mn;

f is 0 or 1;

each R₁ is independently —C(O)OH, —C(O)O⁻ or —C(O)(amino acid residue) or SO₂(amino acid residue); and

n is the number of counterions sufficient to balance the charges of the compound of Formula (B).

In one aspect, the counterion is Cl⁻ or Br⁻.

In one aspect, the amino acid of the amino acid residue is β-alanine, γ-aminobutyric acid, 6-aminohexanoic acid, 5-aminovaleric acid, L-aspartic acid, L-glutamine, L-glutamic acid, glycine, L-phenylalanine, L-tyrosine, or L-valine.

In another aspect, the amino acid residue is L-tyrosine.

In a further embodiment, the metalloporphyrin is selected from

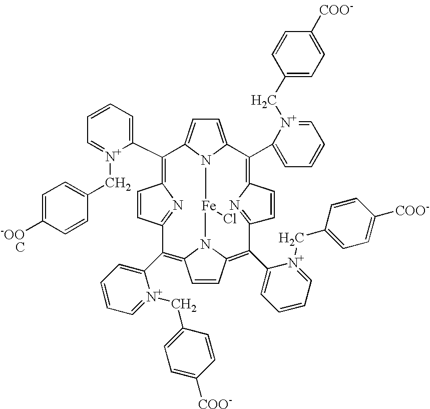

Other features and advantages of the invention will become apparent from the following detailed description, and from the claims.

### BRIEF DESCRIPTION OF THE DRAWINGS

FIGS. 1a and 1b: show graphically the effects of Metalloporphyrin A on plasma concentrations of urea (mmol/L) over time in diabetic rats.

FIGS. 2a and 2b: show graphically the effects of Metalloporphyrin A on plasma concentrations of creatine (μmol/L) over time in diabetic rats.

FIGS. 3a and 3b: show graphically the effects of Metalloporphyrin A on fractional excretion of sodium (FE_{Na}) in diabetic rats.

FIGS. 4a and 4b: show graphically the effects of Metalloporphyrin A on kidney MPO activity in diabetic rats.

FIGS. 5a and 5b: show graphically the effects of Metalloporphyrin A on kidney MDA levels in diabetic rats.

FIG. 6: shows graphically the effects of Metalloporphyrin A on CIN-mediated renal histopathological scoring in diabetic rats.

FIG. 7: shows graphically the effects on plasma NGAL (ng/ml) after saline/placebo administration over time in non-diabetic and diabetic rats.

FIG. 8: shows graphically the effects of Metalloporphyrin A on plasma NGAL (mcg/ml) after Iomeprol administration over time in diabetic rats.

FIG. 9: shows photomicrographs of the effect of Iomeprol on diabetic kidney medulla expression of ICAM-1 using immunohistochemistry and the inhibitory effects on ICAM-1 expression by metalloporphyrin A (1 mg/kg).

FIG. 10: shows photomicrographs of the effect of Iomeprol on diabetic kidney medulla expression of Nitrotyrosine using immunohistochemistry and the inhibitory effects on Nitrotyrosine expression by metalloporphyrin A (1 mg/kg).

FIG. 11: shows photomicrographs of the effect of Iomeprol on diabetic kidney medulla expression of PAR using immunohistochemistry and the inhibitory effects on PAR expression by metalloporphyrin A (1 mg/kg).

FIG. 12: shows graphically the effect of a combination of NAC and metalloporphyrin A on Plasma creatinine levels in a CIN model over time.

FIG. 13: shows graphically the effect of a combination of NAC and metalloporphyrin A on creatinine clearance levels in a CIN model over time.

FIG. 14: shows graphically the effect of a combination of NAC and metalloporphyrin A on urine αGST levels in a CIN model over time.

FIG. 15: shows graphically the effect of a combination of NAC and metalloporphyrin A on total protein levels in a CIN model over time.

FIG. 16: shows graphically the effect of M40403 and a combination of NAC and M40403 on Plasma creatinine levels in a CIN model over time.

FIG. 17: shows graphically the effect of M40403 and a combination of NAC and M40403 on urine protein levels in a CIN model over time.

FIG. 18: shows graphically the effect of M40403 and a combination of NAC and M40403 on Creatinine clearance in a CIN model over time.

FIG. 19: shows graphically the effect of M40403 and a combination of NAC and M40403 on Plasma NGAL levels in a CIN model over time.

FIG. 20: shows graphically the effect of M40403 and a combination of NAC and M40403 on Urine NGAL levels in a CIN model over time.

FIG. 21: shows graphically the effect of M40403 and a combination of NAC and M40403 on Plasma K levels in a CIN model over time.

FIG. 22: shows graphically the effect of M40403 and a combination of NAC and M40403 on Plasma Na levels in a CIN model over time.

FIG. 23: shows graphically the effect of three PARP inhibitors on Plasma Creatinine levels in a CIN model over time.

FIG. 24: shows graphically the effect of three PARP inhibitors Plasma NGAL levels in a CIN model over time.

FIG. 25: shows graphically the effect of three PARP inhibitors on Urine NGAL levels in a CIN model over time.

FIG. 26: shows graphically the effect of three PARP inhibitors on Kidney histological scoring in a CIN model.

FIG. 27: shows graphically the effect of three PARP inhibitors on Urine αGST levels in a CIN model over time.

FIG. 28: shows graphically the effect of PARP inhibitor INO 1001 and a combination of NAC and INO 1001 on plasma creatinine levels in a CIN model.

FIG. 29: shows graphically the effect of PARP inhibitor INO 1001 and a combination of NAC and INO 1001 on creatinine clearance levels in a CIN model.

FIG. 30: shows graphically the effect of PARP inhibitor INO 1001 and a combination of NAC and INO 1001 on plasma NGAL levels in a CIN model.

FIG. 31: shows graphically the effect of PARP inhibitor INO 1001 and a combination of NAC and INO 1001 on urine NGAL levels in a CIN model.

FIG. 32: shows graphically the effect of PARP inhibitor INO 1001 and a combination of NAC and INO 1001 on urine αGST levels in a CIN model.

FIG. 33: shows graphically the effect of PARP inhibitor INO 1001 and a combination of NAC and INO 1001 on urine protein levels in a CIN model.

### DETAILED DESCRIPTION OF THE INVENTION

In order that the present invention may be more readily understood, certain terms are first defined. Additional definitions are set forth throughout the detailed description.

### I. DEFINITIONS

The term “contrast-induced nephropathy” (CIN) is to be understood as existing upon a 25% relative increase in serum creatinine levels within 24-72 hours of contrast agent administration in a given subject in the absence of other attributing factors.

The term “peroxynitrite decomposition agent” is to be understood to include a compound that reacts directly to decompose peroxynitrite and to attenuate the toxic effects of peroxynitrite. Such compounds include, but are not limited to metalloporphyrins of iron and manganese.

The term “contrast agent” is to be understood as including compounds that are used to improve the visibility of internal bodily structures in an X-ray or MRI image. Such compounds include, but are not limited to:
High osmolar agents, such as Iothalamate, Metrizoate and Diatrizoate; where the osmolality of these agents is about 5 times greater than the osmolality of blood;
Low osmolar agents, such as Iohexyl, Ioversol, Iopamidol, Iopromide, Iomeprol and Ioxaglate, Ioxilan; where the osmolality of these agents is about 2-3 times greater than the osmolality of blood; and
Iso-osmolar agents such as Iotrolan and Iodixanol; where the osmolality of these agents is the same as the osmolality of blood.

The terms “prevent” or “prevention,” as used herein, refer to preventative measures described herein. The methods of “prevention” employ administration to a subject, a metalloporphyrin of the present invention and/or a SOD mimic, for example, a subject at risk of developing CIN or predisposed to CIN, in order to prevent, reduce the severity of, or ameliorate one or more symptoms of CIN in order to prolong the outcomes or survival of a subject beyond that expected in the absence of such administration.

The terms “effective amount” and “therapeutically effective amount,” as used herein, refers to that amount of a peroxynitrite decomposition agent and/or a SOD mimic that is sufficient to mitigate, reduce or prevent the effects of contrast induced nephropathy after administration of contrast media to a subject. A therapeutically effective amount will vary depending upon the subject and the risk factors of the subject, such as the weight, age, presence or absence of diabetes and renal sufficiency, of the subject, the volume of contrast agent involved, the manner of administration and the like, which can readily be determined by one of ordinary skill in the art. The dosages for administration can range from, for example, about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg, of a peroxynitrite decomposition agent and/or a SOD mimic of the present invention. Dosage regimens may be adjusted to provide the optimum therapeutic response. An effective amount is also one in which any toxic or detrimental effects (i.e., side effects) of a given peroxynitrite decomposition agent and/or a SOD mimic are minimized and/or outweighed by the beneficial effects.

As used herein, the term “subject” includes any human or non-human animal. For example, the methods and compositions of the present invention can be used to treat a subject at risk of developing contrast-induced nephropathy. In a particular embodiment, the subject is a human. The term “non-human animal” includes all vertebrates, e.g., mammals and non-mammals, such as non-human primates, sheep, dog, cow, chickens, amphibians, reptiles, etc.

Illustrative “counterions” include but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, pamoate (i.e., 1,1′-methylene-bis-(2-hydroxy-3-naphthoate)), camphorsulfonate, 2-methylbenzoate, 3-methylbenzoate, and 4-methylbenzoate counterions.

A “calcium channel blocker”, is to be understood as including, but not limited to, Amlodipine, Felodipine, Nicardipine, Nifedipine, Nimodipine, Nisoldipine, Nitrendipine, Lacidipine, Lercanidipine, Verapamil, Gallopamil, Diltiazem, and Menthol.

An “ACE inhibitor,” is to be understood as including, but not limited to, Captopril, Zofenopril, Enalapril, Ramipril, Quinapril, Perindopril, Lisinopril, Benazepril, and Fosinopril.

A PARP inhibitor is to be understood as a compound that is capable of binding to and inhibiting the nuclear enzyme poly(ADP-ribose) polymerase (PARP) thereby inhibiting PARP-mediated repair of single strand DNA breaks. Such compounds include INO 1001, PJ34, ABT888, AG14699, AG14361, KU59346, BSI 201 and GPI 21016.

An adenosine antagonist is to be understood as a compound that binds to the adenosine receptor and acts as an antagoinist to the adenosine receptor, including, but not limited to, KW6002 and SCH-58261, theophylline, MRSI191, MRS1523 and MRE3008F20.

A superoxide dismutase mimic (SOD mimic) is to be understood to include a compound that acts as an oxidoreductase directly with superoxide (O₂⁻) to attenuate O₂⁻ mediated cell injury. Such compounds include, but are not limited to, metalloporphyrins of iron, copper and manganese, such as manganese tetrakis (4-benzoic acid) porphyrin (MnTBAP), M40403, M40419 and AEOL-10113.

Other embodiments of the present invention are described in the following Examples.

The present invention is further illustrated by the following examples that should not be construed as further limiting. The contents of figures and all references, patents and published patent applications cited throughout this application are expressly incorporated herein by reference.

### EXAMPLES

Various aspects of the invention are described in further detail in the following subsections. Illustrative peroxynitrite decomposition agents of the present invention are shown below:

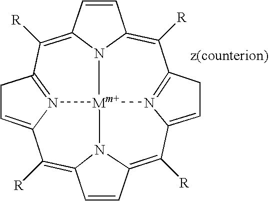

wherein each R is selected from the following

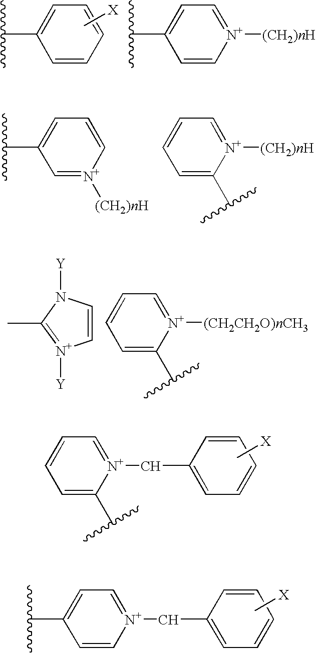

where X is selected from —C(O)OH, —C(O)O⁻ or —C(O)(amino acid residue), SO₂OH, SO₂O⁻ or SO₂(amino acid residue);

where each Y is independently selected from halogen, C₁-C₆ alkyl, C₁-C₆ alkyl-O—C₁-C₆ alkyl,

each n is independently an integer from 1 to 4.

Specific peroxynitrite decomposition agents/metalloporphyrins that are illustrative of the invention are represented in the following Table:

| Metalloporphyrin | M | R | X | n |
|---|---|---|---|---|
| A | FeCl | 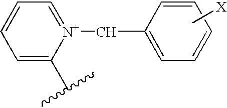 | —C(O)O⁻ | — |
| B | FeBr | 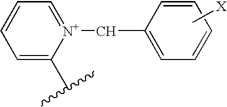 | —C(O)O⁻ | — |
| C | FeOAc | 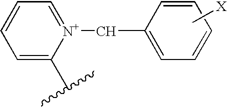 | | — |
| D | Fe-2-methylbenzoate | 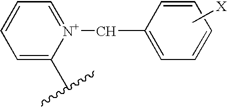 | | — |
| E | MnCl | 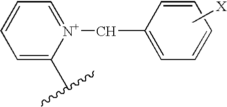 | —C(O)O⁻ | — |
| F | MnBr | 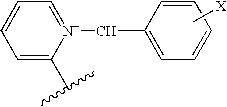 | —C(O)O⁻ | — |
| G | MnOAc | 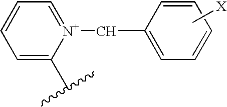 | —C(O)O⁻ | — |
| H | Mn-2-methylbenzoate | 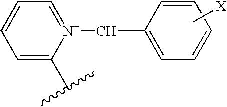 | —C(O)O⁻ | — |
| I | Mn-2-methylbenzoate | 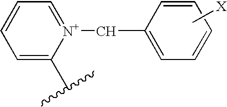 | —C(O)O⁻ | — |
| J | Mn | 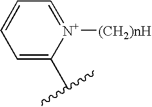 | — | 1 |
| K | Mn | 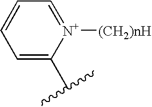 | — | 2 |
| L | Mn | 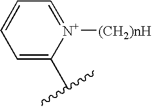 | — | 4 |
| M | FeOAc | 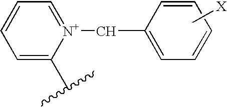 | —(CO)NHCH₂—COOH | — |
| N | FeCl | 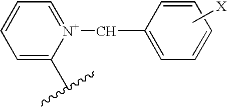 | —(CO)NHCH₂—COOH | — |
| 0 | FeOAc | 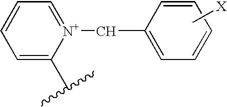 | —(SO₂)NHCH₂—COOH | — |
| P | Mn | 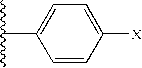 | —C(O)OH | — |
| Q | Fe | 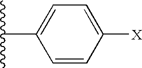 | —SO₃⁻ | — |
| R | Mn | 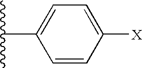 | —SO₃⁻ | — |
| S | Mn | 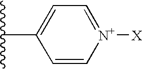 | —CH₃ | — |
| T | Fe | 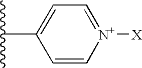 | —CH₂CH₃ | — |
| U | Mn | 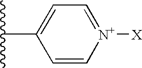 | —CH₂CH₃ | — |
| V | Mn | 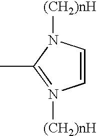 | — | 2 |
| W | Mn | 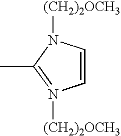 | — | — |
| X | FeCl | 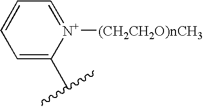 | — | 3 |

### Synthetic Methods

A. Synthetic methods for producing Metalloporphyrins A to I are described in detail in US 2006/0003982.
B. Synthetic methods for producing Metalloporphyrins J to L, P, S and U are described in detail in U.S. Pat. No. 6,916,799.
C. Synthetic methods for producing Metalloporphyrins M to 0 are described in detail in WO2007/038630.
D. Synthetic methods for producing Metalloporphyrin V and several other related metalloporphyrins are described in detail in U.S. Pat. No. 6,544,975.
E. Synthetic methods for producing Metalloporphyrin V are described in detail in U.S. Pat. No. 6,544,975.
F. Metalloporphyrin Q can be obtained from Calbiochem (La Jolla, Calif.)
G. Synthetic methods for producing Metalloporphyrin T are described in J. Inorg. Nucl. Chem. 1977, 39, 1865-1870 and U.S. Pat. No. 6,969,707.
H. The synthesis of Metalloporphyrin X is described in Szabó C, Mabley J G, Moeller S M, et al. Mol. Med. 2002; 8:571-580.

It is to be appreciated that many of the Metalloporphyrins defined above can exist in different isomeric forms. For example, the metalloporphyrins A-H and M to O contain four pyridyl groups. Due to steric factors, each pyridyl group's nitrogen atom can exist: (1) above the plane of the porphyrin ring (this conformation is herein referred to as the [beta]-position); or (2) below the plane of the porphyrin ring (this conformation is herein referred to as the [alpha]-position). In one embodiment, a metalloporphyrin is substantially free of its other atropisomers. In another embodiment, a metalloporphyrin of the invention exists as a mixture of two or more isomers.

It is to be further appreciated that the Metalloporphyrins defined above when in solid state or when in vivo may form a hydrate or an aquo complex through association or co-ordination with one or more molecules of water.

It is to be further appreciated that a counterion or water molecule that forms a bond with M as defined above for the Metalloporphyrins can exist above or below the plane of the porphyrin ring.

Illustrative superoxide dismutase mimics (SOD mimic), are shown below:

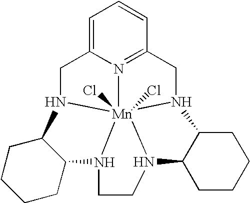

M40403, the synthesis and purification of which is described in WO2002/071054.

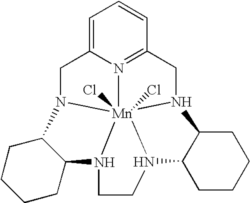

M40419, the synthesis and purification of which is described in WO2002/071054.

Manganese tetrakis (4-benzoic acid) porphyrin (MnTBAP), the synthesis of which is described in U.S. Pat. No. 6,916,799.

AEOL 10113 (TE-2-PyP) is described in U.S. Pat. No. 6,916,799.

A number of patents and patent applications describe PARP inhibitors and methods of preparing PARP inhibitors, such as U.S. Pat. Nos. 6,828,319, 6,956,035, 7,381,722, PCT/US2008/055361 and PCT/US2006/033018.

When administered to a subject, the peroxynitrite decomposition agent, SOD mimic and/or PARP inhibitor can be administered as a component of a composition that comprises a physiologically acceptable carrier or vehicle. The present compositions, which comprise a peroxynitrite decomposition agent, SOD mimic and/or PARP inhibitor, can be administered orally. The peroxynitrite decomposition agent, SOD mimic and/or PARP inhibitor of the invention can also be administered by any other convenient route, for example, by infusion or bolus injection, by absorption through epithelial or mucocutaneous linings (e.g., oral, rectal, and intestinal mucosa) and can be administered together with another biologically active agent. Administration can be systemic or local. Various delivery systems are known, e.g., encapsulation in liposomes, microparticles, microcapsules, capsules, and can be administered.

Methods of administration include, but are not limited to, intradermal, intramuscular, intraperitoneal, intravenous, ocular, subcutaneous, intranasal, epidural, oral, sublingual, intracerebral, intravaginal, transdermal, rectal, by inhalation, or topical, particularly to the ears, nose, eyes, or skin. In some instances, administration will result in the release of the metalloporphyrins into the bloodstream. The mode of administration can be left to the discretion of the practitioner.

In one embodiment, the peroxynitrite decomposition agent, SOD mimic and/or PARP inhibitor is administered orally.

In other embodiments, it can be desirable to administer the peroxynitrite decomposition agent, SOD mimic and/or PARP inhibitor locally. This can be achieved, for example, and not by way of limitation, by local infusion during surgery, by means of a catheter, by means of a suppository or enema, or by means of an implant, said implant being of a porous, non-porous, or gelatinous material, including membranes, such as sialastic membranes, or fibers.

In certain embodiments, it can be desirable to introduce the peroxynitrite decomposition agent, SOD mimic and/or PARP inhibitor into the central nervous system or gastrointestinal tract by any suitable route, including intraventricular, intrathecal, and epidural injection, and enema. Intraventricular injection can be facilitated by an intraventricular catheter, for example, attached to a reservoir, such as an Ommaya reservoir.

In certain embodiments, the peroxynitrite decomposition agent, SOD mimic and/or PARP inhibitor can be formulated as a suppository, with traditional binders and excipients such as triglycerides.

In another embodiment the peroxynitrite decomposition agent and/or SOD mimic can be delivered in a vesicle, in particular a liposome (see Langer, Science 249:1527-1533 (1990) and Treat or prevent et al., Liposomes in the Therapy of Infectious Disease and Cancer 317-327 and 353-365 (1989)).

In yet another embodiment the peroxynitrite decomposition agent, SOD mimic and/or PARP inhibitor can be delivered in a controlled-release system or sustained-release system (see, e.g., Goodson, in Medical Applications of Controlled Release, supra, vol. 2, pp. 115-138 (1984)). Other controlled or sustained-release systems discussed in the review by Langer, Science 249:1527-1533 (1990) can be used. In one embodiment, a pump can be used (Langer, Science 249:1527-1533 (1990); Sefton, CRC Crit. Ref. Biomed. Eng. 14:201 (1987); Buchwald et al., Surgery 88:507 (1980); and Saudek et al., N. Engl. J. Med. 321:574 (1989)). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

In yet another embodiment, a controlled- or sustained-release system can be placed in proximity of a target of the peroxynitrite decomposition agent, SOD mimic and/or PARP inhibitor, e.g., the kidney, thus requiring only a fraction of the systemic dose.

The present compositions can optionally comprise a suitable amount of a pharmaceutically acceptable excipient so as to provide the form for proper administration to the subject.

Such pharmaceutical excipients can be liquids, such as water and oils, including those of petroleum, animal, vegetable, or synthetic origin, such as peanut oil, soybean oil, mineral oil, sesame oil and the like. The pharmaceutical excipients can be saline, gum acacia, gelatin, starch paste, talc, keratin, colloidal silica, urea and the like. In addition, auxiliary, stabilizing, thickening, lubricating, and coloring agents can be used. In one embodiment the pharmaceutically acceptable excipients are sterile when administered to a subject. Water is a particularly useful excipient when the metalloporphyrin is administered intravenously. Saline solutions and aqueous dextrose and glycerol solutions can also be employed as liquid excipients, particularly for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment the composition is in the form of a capsule (see e.g. U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

In one embodiment the peroxynitrite decomposition agent, SOD mimic and/or PARP inhibitor is/are formulated in accordance with routine procedures as a composition adapted for oral administration to human beings. Compositions for oral delivery can be in the form of tablets, lozenges, aqueous or oily suspensions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, when in tablet or pill form, the compositions can be coated to delay disintegration and absorption in the gastrointestinal tract thereby providing a sustained action over an extended period of time. Selectively permeable membranes surrounding an osmotically active driving the peroxynitrite decomposition agent, SOD mimic and/or PARP inhibitor are also suitable for orally administered compositions. In these latter platforms, fluid from the environment surrounding the capsule is imbibed by the driving compound, which swells to displace the agent or agent composition through an aperture. These delivery platforms can provide an essentially zero-order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment, the excipients are of pharmaceutical grade.

In another embodiment, the peroxynitrite decomposition agent, SOD mimic and/or PARP inhibitor can be formulated for intravenous administration. Typically, compositions for intravenous administration comprise sterile isotonic aqueous buffer.

Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized-powder or water free concentrate in a hermetically sealed container such as an ampule or sachette indicating the quantity of active agent. Where the peroxynitrite decomposition agent, SOD mimic and/or PARP inhibitor are to be administered by infusion, they can be dispensed, for example, with an infusion bottle containing sterile pharmaceutical grade water or saline. Where peroxynitrite decomposition agent, SOD mimic and/or PARP inhibitor are administered by injection, an ampule of sterile water for injection or saline can be provided so that the ingredients can be mixed prior to administration.

The peroxynitrite decomposition agent, SOD mimic and/or PARP inhibitor can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release.

In one embodiment, a controlled- or sustained-release composition comprises a minimal amount of a peroxynitrite decomposition agent, SOD mimic and/or PARP inhibitor to prevent the development of CIN. Advantages of controlled- or sustained-release compositions include extended activity of the drug, reduced dosage frequency, and increased subject compliance. In addition, controlled- or sustained-release compositions can favorably affect the time of onset of action or other characteristics, such as blood levels of peroxynitrite decomposition agent, SOD mimic and/or PARP inhibitor, and can thus reduce the occurrence of adverse side effects.

Controlled- or sustained-release compositions can initially release an amount of peroxynitrite decomposition agent, SOD mimic and/or PARP inhibitor that promptly produces the desired therapeutic or prophylactic effect, and gradually and continually release other amounts of peroxynitrite decomposition agent, SOD mimic and/or PARP inhibitor mimic to maintain this level of therapeutic or prophylactic effect over an extended period of time. To maintain a constant level of peroxynitrite decomposition agent, SOD mimic and/or PARP inhibitor in the body, the peroxynitrite decomposition agent, SOD mimic and/or PARP inhibitor can be released from the dosage form at a rate that will replace the amount of metalloporphyrin being metabolized and/or excreted from the body. Controlled- or sustained-release of an active ingredient can be stimulated by various conditions, including but not limited to, changes in pH, changes in temperature, concentration or availability of enzymes, concentration or availability of water, or other physiological conditions or compounds.

The amount of peroxynitrite decomposition agent, SOD mimic and/or PARP inhibitor that is effective in the prevention of CIN can be determined by standard clinical techniques. In addition, in vitro or in vivo assays can optionally be employed to help identify optimal dosage ranges. The precise dose to be employed can also depend on the route of administration, the time of the subject's exposure to contrast media, the amount of contrast media that a subject is exposed to, or the seriousness of CIN being prevented or treated. In one embodiment the effective dosage is about 0.01 mg, 0.5 mg, about 1 mg, about 50 mg, about 100 mg, about 200 mg, about 300 mg, about 400 mg, about 500 mg, about 600 mg, about 700 mg, about 800 mg, about 900 mg, about 1 g, about 1.2 g, about 1.4 g, about 1.6 g, about 1.8 g, about 2.0 g, about 2.2 g, about 2.4 g, about 2.6 g, about 2.8 g, about 3.0 g, about 3.2 g, about 3.4 g, about 3.6 g, about 3.8 g, about 4.0 g, about 4.2 g, about 4.4 g, about 4.6 g, about 4.8 g, and about 5.0 g, every 4 hours.

Equivalent dosages may be administered over various time periods including, but not limited to, about every 2 hours, about every 6 hours, about every 8 hours, about every 12 hours, about every 24 hours, about every 36 hours, about every 48 hours or about every 72 hours. The effective dosage amounts described herein refer to total amounts administered; that is, if more than one peroxynitrite decomposition agent, SOD mimic and/or PARP inhibitor is administered, the effective dosage amounts correspond to the total amount administered.

When the peroxynitrite decomposition agent, SOD mimic and/or PARP inhibitor are administered for prevention of CIN, the peroxynitrite decomposition agent, SOD mimic and/or PARP inhibitor can be administered within 48 hours prior to administration of the contrast media. Subsequent administration may be repeated at regular intervals as set forth above.

In one embodiment, an initial dose of the peroxynitrite decomposition agent, SOD mimic and/or PARP inhibitor is administered from about 5 minutes to about one hour prior to exposure to contrast media with repeated doses optionally administered at regular intervals thereafter.

The peroxynitrite decomposition agent, SOD mimic and/or PARP inhibitor can be assayed in vitro or in vivo for the desired therapeutic or prophylactic activity prior to use in humans. Animal model systems can be used to demonstrate safety and efficacy.

The present methods for preventing CIN in a subject at risk thereof can further comprise administering another therapeutic agent, such as NAC to the subject being administered the peroxynitrite decomposition agent, SOD mimic and/or PARP inhibitor.

In one embodiment the other therapeutic agent is administered in an effective amount.

Effective amounts of the other therapeutic agents are well known to those skilled in the art. However, it is well within the skilled artisan's purview to determine the other therapeutic agent's optimal effective amount range. In one embodiment of the invention, where another therapeutic agent is administered to a subject, the effective amount of the peroxynitrite decomposition agent, SOD mimic and/or PARP inhibitor is less than its effective amount would be where the other therapeutic agent is not administered. In this case, without being bound by theory, it is believed that the peroxynitrite decomposition agent, SOD mimic and/or PARP inhibitor act together to prevent CIN.

### CIN Animal Model

Male Wistar rats (150-200 g; Harlan Nossanr) were housed in a controlled environment and provided with standard rodent chow and water.

### Induction of Diabetes

After 12 hours of fasting, the animals received a single 60 mg/kg intravenous (i.v.) injection of streptozotocin (Sigma, St. Louis, Mo.) in 10 mM sodium citrate buffer, pH 4.5 (see figure showing CIN model). Control non-diabetic animals were fasted and received citrate buffer alone. After 24 hours, animals with blood glucose levels greater than 250 mg/dl were considered diabetic. All experiments were performed 10 days following the induction of diabetes. The diabetic state was evaluated daily by determination of the blood glucose levels. Upon the induction of the diabetic state the animals were presenting with a major risk factor pre-disposing the animals to CIN after the administration of contrast media.

### Contrast-Induced Nephropathy (CIN)

Ten days following the induction of diabetes the rats were anesthetized with 90 mg/kg ketamine i.m. and 10 mg/kg xylazine i.m. and were treated with the contrast agent iomeprol (10 mL/kg injected via the lateral tail vein) or with 0.9% normal saline. The contrast agent used was the low osmolar non-ionic monomer iomeprol (Iomeron, 400 mgI mL-1; Bracco SpA, Milan, Italy) with an osmolality of 726±34 mosmol/kg H₂O. Upon completion of surgical procedures, the animals were randomly allocated to eleven different experimental groups as tabulated below:

### Example 1

### Effect of Metalloporphyrin A on CIN

The following groups of animals were prepared and studied as follows:

| | | TREATMENT | | | | | | |
|---|---|---|---|---|---|---|---|---|
| | | | | | Daily | Stock | Dose | Dosing |
| Group | Individual | Drug/Test | | | Dose | Solution of | Volume | Frequency |
| No. | Animal No. | Material | Lot # | Route | (mg/kg) | Drug (mg/ml) | (ml/animal) | & duration |
| 1 | Diabetic rats | Saline | None | i.p. | None | None | None | Saline 2X |
| | (n = 8) | | | | | | | per day |
| | +i.v. SALINE | | | | | | | for 4 days |
| | (10 ml/kg) | | | | | | | |
| 2 | Diabetic rats | +Metallo- | MC- | i.p. | 1 | 0.25 | 0.25 | 2X per day |
| | (n = 8) | porphyrin A | 016-91 | | | | | for 4 days |
| | +i.v. SALINE | | | | | | | |
| | (10 ml/kg) | | | | | | | |
| 3 | Diabetic rats | Saline | None | i.p. | None | 0.9% NaCl | 0.25 | Saline 2X |
| | (n = 8) | | | | | | | per day |
| | +i.v. | | | | | | | for 4 days |
| | IOMEPROL | | | | | | | |
| | (10 ml/kg) | | | | | | | |
| 4 | Diabetic rats | +Metallo- | MC- | i.p. | 0.03 | 0.25 | 0.25 | 2X per day |
| | (n = 8) | porphyrin A | 016-91 | | | | | for 4 days |
| | +i.v. | | | | | | | |
| | IOMEPROL | | | | | | | |
| | (10 ml/kg) | | | | | | | |
| 5 | Diabetic rats | +Metallo- | MC- | i.p. | 0.1 | 0.25 | 0.25 | 2X per day |
| | (n = 8) | porphyrin A | 016-91 | | | | | for 4 days |
| | +i.v. | | | | | | | |
| | IOMEPROL | | | | | | | |
| | (10 ml/kg) | | | | | | | |
| 6 | Diabetic rats | +Metallo- | MC- | i.p. | 0.3 | 0.25 | 0.25 | 2X per day |
| | (n = 8) | porphyrin A | 016-91 | | | | | for 4 days |
| | +i.v. | | | | | | | |
| | IOMEPROL | | | | | | | |
| | (10 ml/kg) | | | | | | | |
| 7 | Diabetic rats | +Metallo- | MC- | i.p. | 1 | 0.25 | 0.25 | 2X per day |
| | (n = 8) | porphyrin A | 016-91 | | | | | for 4 days |
| | +i.v. | | | | | | | |
| | IOMEPROL | | | | | | | |
| | (10 ml/kg) | | | | | | | |
| 8 | Diabetic rats | +NAC | R05CB01 | i.p. | 10 | 100 mg/ml | 0.25 | 1X per day |
| | (n = 8) | | | | | | | for 4 days |
| | +i.v. SALINE | | | | | | | |
| | (10 ml/kg) | | | | | | | |
| 9 | Diabetic rats | NAC | R05CB01 | i.p. | 10 | 100 mg/ml | 0.25 | 1X per day |
| | (n = 8) | | | | | | | for 4 days |
| | +i.v. | | | | | | | |
| | IOMEPROL | | | | | | | |
| | (10 ml/kg) | | | | | | | |
| 10 | Wild-type rats | +saline | None | i.p. | None | 0.9% NaCl | 0.25 | 1X per day |
| | (n = 8) | | | | | | | for 4 days |
| | +i.v. SALINE | | | | | | | |
| | (10 ml/kg) | | | | | | | |
| 11 | Wild-type rats | +saline | None | i.p. | None | 0.9% NaCl | 0.25 | 1X per day |
| | (n = 10) | | | | | | | for 4 days |
| | +i.v. | | | | | | | |
| | IOMEPROL | | | | | | | |
| | (10 ml/kg) | | | | | | | |

Experimental model for assessing the effects of Metalloporphyrin A on CIN: Diabetic (n=8) for group #'s 1-9 or wild-type rats (n=10) for group #'s 10 and 11 were treated with drug 60 min prior to contrast agent administration. At the “0” time point, Iomeprol was administered with ensuing drug intervention dosing every 12 hours for Metalloporphyrin A from 24-84 hours or every 24 hours for NAC from 24-72 hours. At the 96 hour time point, kidneys were harvested for histopathology, MPO and MDA measurements. From 0-96 hours, plasma samples from all animals per group were screened for urea, creatinine, fractional excretion of Na⁺ and NGAL.

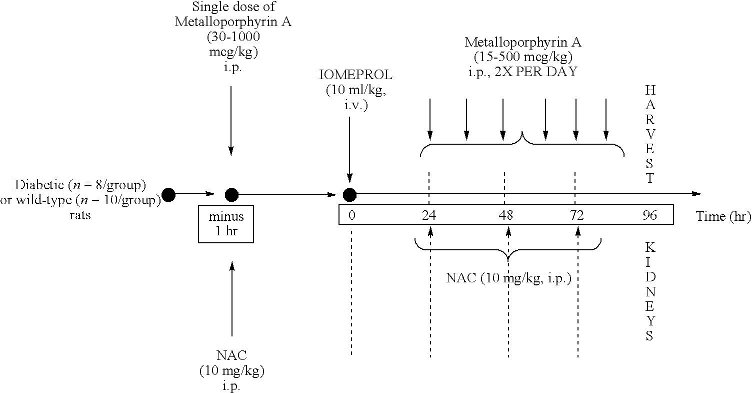

### Measurement of Biochemical Parameters

At the indicated time point blood samples were collected via the lateral tail vein into S1/3 tubes containing serum gel. The samples were centrifuged (6000 r.p.m. for 3 min) to separate plasma. All plasma samples were analyzed for biochemical parameters within 24 hours of collection. Plasma concentrations of urea and creatinine were measured as indicators of impaired glomerular function. Urine samples were collected at 96 hour after contrast agent administration and the volume of urine produced was recorded. Urine concentrations of Na⁺ were measured and were used in conjunction with plasma Na⁺ concentrations to calculate fractional excretion of Na⁺ (FE_{Na}) using standard formulae, which was used as an indicator of tubular function.

### Determination of Myeloperoxidase (MPO) Activity

Myeloperoxidase (MPO) activity in kidneys was used as an indicator of polymorphonuclear (PMN) cell infiltration activation using a method previously described (Hillegass et al, J. Pharmacol. Methods. 24(4):285-95,1990).

At the end of the experiments, kidney tissue was weighed and homogenized in a solution containing 0.5% (wt/vol) hexadecyltrimethylammonium bromide dissolved in 10 mmol/L potassium phosphate buffer (pH 7.4) and centrifuged for 30 minutes at 20,000 g at 4° C. An aliquot of supernatant was then removed and added to a reaction mixture containing 1.6 mmol/L tetramethylbenzidine and 0.1 mmol/L hydrogen peroxide (H₂O₂). The rate of change in absorbance was measured spectrophotometrically at 650 nm. MPO activity was defined as the quantity of enzyme required to degrade 1 mmol of H₂O₂ at 37° C. and was expressed in U/g wet tissue.

### Determination of Malondialdehye Levels

Levels of malondialdehyde (MDA) in kidneys were determined as an indicator of lipid peroxidation following a protocol described previously (Davenport et. al. Clin. Transplant. 9 (3 Pt 1):171-51995).

Kidney tissue was weighed and homogenized in a 1.15% (wt/vol) KCl solution. A 100 mL aliquot of homogenate was then removed and added to a reaction mixture containing 200 mL 8.1% (wt/vol) lauryl sulfate, 1.5 mL 20% (vol/vol) acetic acid (pH 3.5), 1.5 mL 0.8% (wt/vol) thiobarbituric acid, and 700 mL distilled water. Samples were then boiled for one hour at 95° C. and centrifuged at 3000 g for 10 minutes. The absorbance of the supernatant was measured spectrophotometrically at 650 nm. MDA levels were expressed as μM/100 mg wet tissue.

### Histologic Evaluation

At postmortem, a 5 μm section of kidney was removed and placed in formalin and processed through to wax. Five millimeter sections were cut and stained with hematoxylin and eosin. Histologic assessment of tubular necrosis was determined semi-quantitatively using a method modified from McWhinnie et al, Transplantation. 42(4):352-81986, 1986.

Histologic assessment of outer medulla damage was examined by an experienced morphologist, who was not aware of the sample identity. The criteria for injury/necrosis were the following: 0=normal histology; 1=minor edema, minor cell swelling; 2=haemorrhage, moderate edema, moderate cells vacuolization and swelling; 3=moderate haemorrhage, moderate edema, moderate cells vacuolization, swelling and chromatin alteration; 4=severe edema, severe cells vacuolization, swelling and chromatin alteration, presence of necrosis spot; 5=severe edema, severe cells vacuolization, swelling and chromatin alteration, severe necrosis.

### Immunohistochemical Localization of ICAM-1, Nitrotyrosine and Poly(ADP-Ribose)

Rat kidneys fixed in 10% (wt/vol) neutral buffered paraformaldehyde and 8 mm sections were prepared from paraffin-embedded tissues. After deparaffination, endogenous peroxidase was quenched using 0.3% (vol/vol) H₂O₂ in 60% methanol for 30 minutes. The sections were permeabilized using 0.1% (wt/vol) Triton X-100 in phosphate-buffered saline (PBS; 0.01 mol/L, pH 7.4) for 20 minutes. Nonspecific adsorption was minimized by incubating sections in 2% (vol/vol) normal goat serum in PBS for 20 minutes. Endogenous avidin- and biotin-binding sites were blocked by sequential incubation for 15 minutes with avidin (DBA, Milan, Italy) and biotin (DBA, Milan, Italy), respectively. The sections were then incubated overnight with primary antinitrotyrosine antibody (1:1000), anti-PAR antibody (1:500) and anti-ICAM-1 antibody (1:500). Separate sections were also incubated with control solutions consisting of PBS alone or a 1:500 dilution of nonspecific purified rabbit IgG (DBA). Specific labeling was detected using a biotin-conjugated goat antirabbit IgG (DBA) and avidin-biotin peroxidase (DBA). Samples were then viewed under a light microscope.

### Materials

Unless otherwise stated, all compounds were obtained from Sigma-Aldrich Company Ltd. (Milan, Italy). All stock solutions were prepared in non-pyrogenic saline (0.9% NaCl; Baxter, Italy) or 10% DMSO.

### Results

### A. Effect of Metalloporphyrin A on Contrast Agent (Iomeprol)-Mediated Glomerular Dysfunction

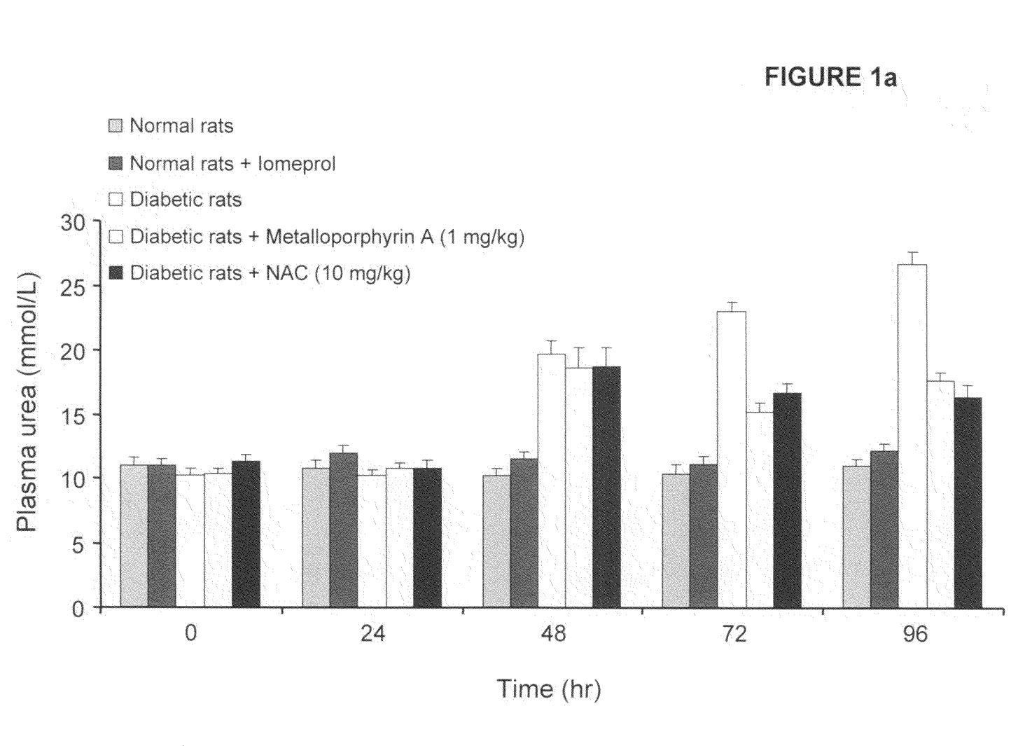

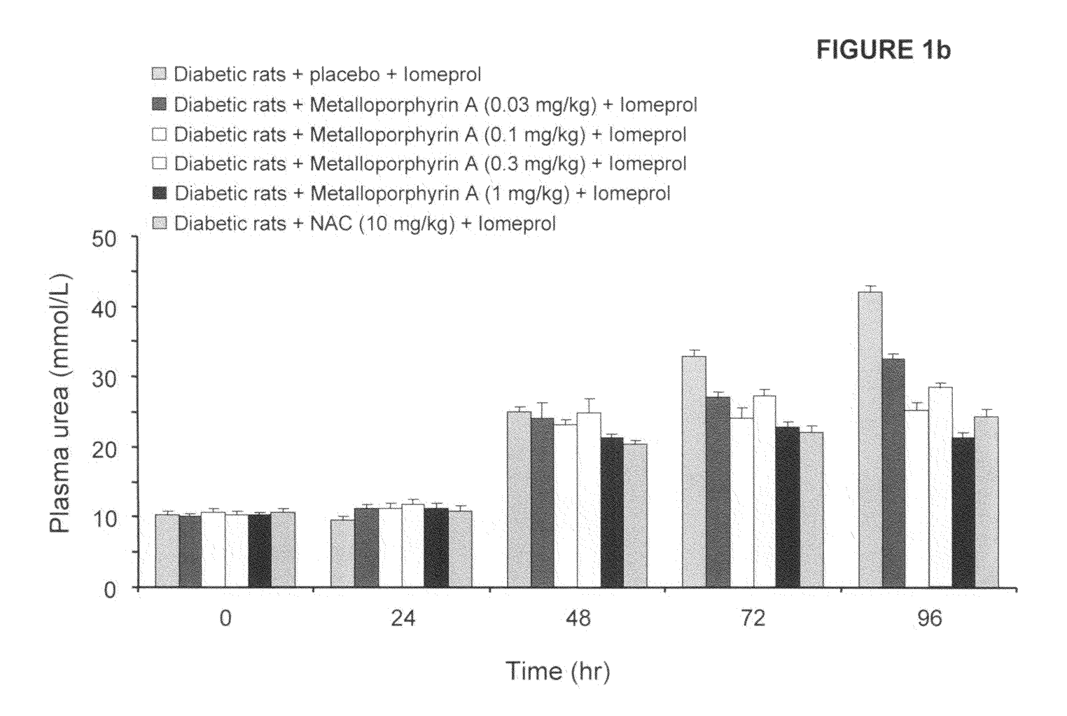

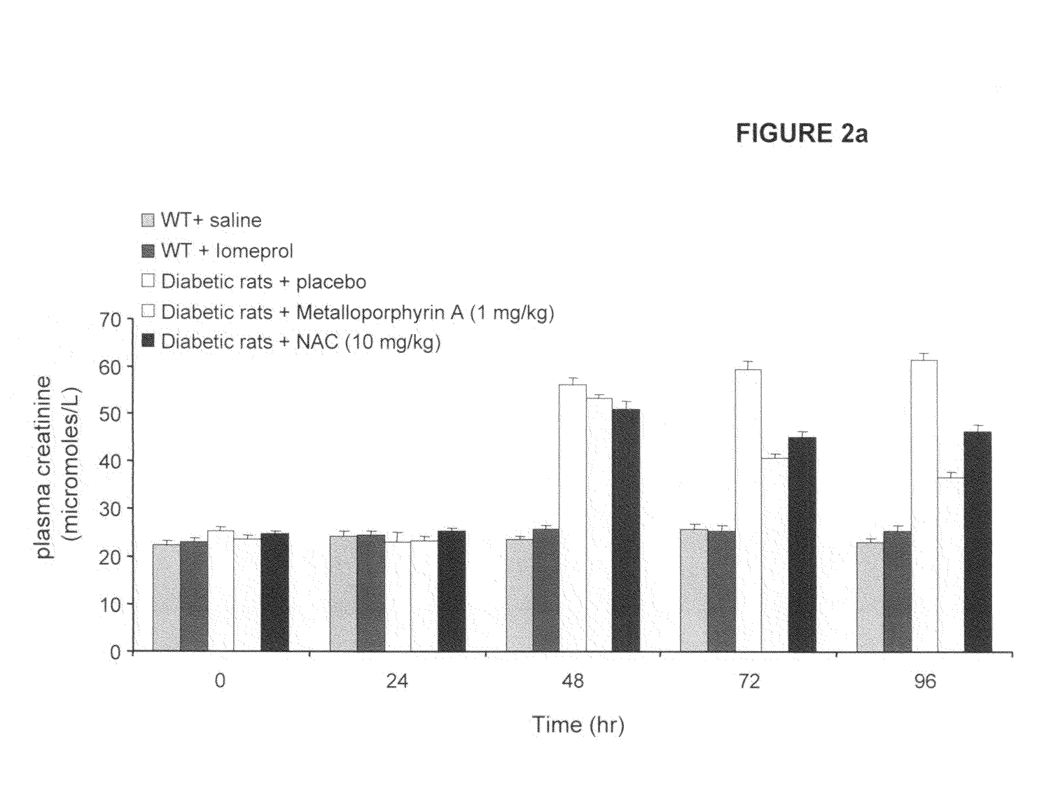

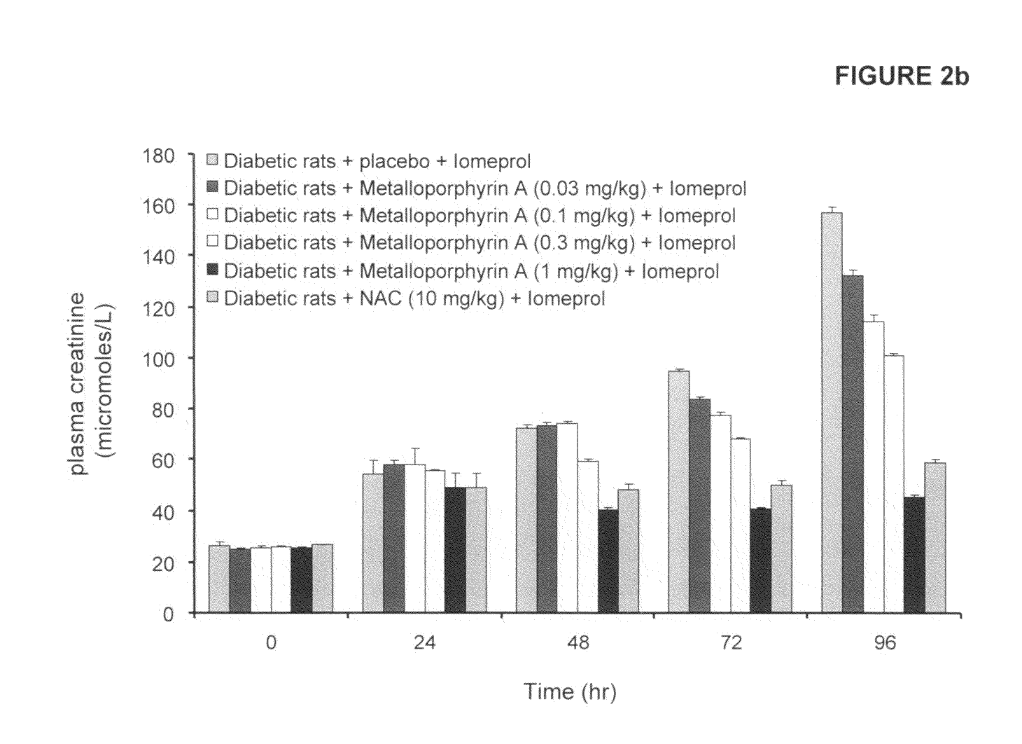

Diabetic rats exhibited a significant increase in the plasma concentrations of urea (FIG. 1a) and creatinine (FIG. 2a) at 72 and 96 hours after the administration of saline in comparison to non-diabetic animals (FIG. 1a). Metalloporphyrin A (at the dose of 1000 μg/kg) produced significant reductions in plasma urea (FIG. 1a) and creatinine (FIG. 2a) concentrations at 48, 72 and 96 hours in diabetic animals. Similarly, the treatment with NAC (10 mg/kg) significantly reduced the increase in the plasma concentrations of urea (FIG. 1a) and creatinine (FIG. 2a) in diabetic rats at 72 and 96 hours. In addition, a significant increase of the plasma concentrations of urea (FIG. 1b) and creatinine (FIG. 2b) was observed in diabetic rats at 24, 48, 72 and 96 hours after the administration of the contrast agent. Metalloporphyrin A (at the dose of 1000 μg/kg) produced significant reductions in plasma urea (FIG. 1b) and creatinine (FIG. 2b) concentrations at 48, 72 and 96 hours. Metalloporphyrin A (at the doses of 30-300 μg/kg) treatment significantly reduced the plasma urea (FIG. 1b) and creatinine (FIG. 2b) concentrations at 72 and 96 hours. Similarly, the treatment with NAC (10 mg/kg) produced reductions in plasma urea (FIG. 1b) and creatinine (FIG. 2b) concentrations at 48, 72 and 96 hours.

### B. Effect of Metalloporphyrin A on CIN-Mediated Tubular Dysfunction and Injury

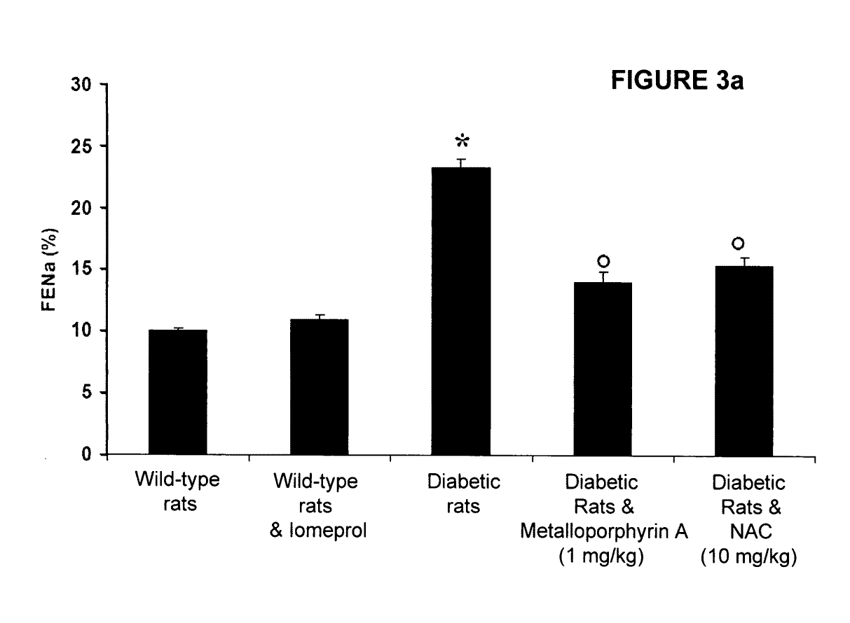

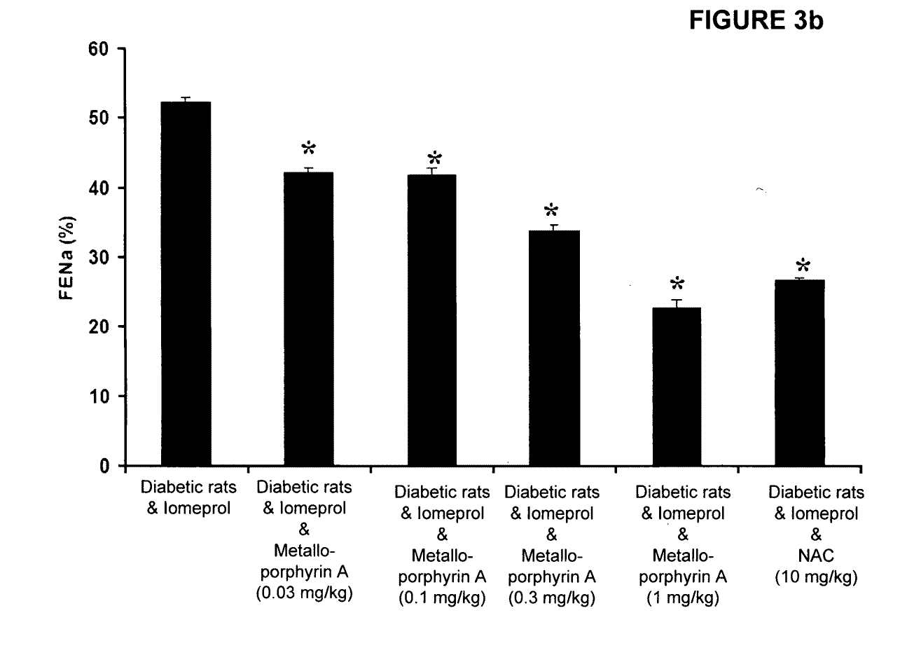

Fractional excretion of sodium, calculated using plasma Na⁺ concentrations, urine production (urine flow, mL/min) and urinary concentrations of Na⁺, was used as an indicator of proximal tubule (PT) function. A significant increase in FE_{Na} (FIG. 3a) was observed in diabetic rats at 96 hours after the administration of saline in comparison to non diabetic animals (FIG. 3a). Metalloporphyrin A (at the dose of 1000 μg/kg) produced significant reductions in in FE_{Na} at 96 hours in diabetic animals. Similarly, the treatment with NAC (10 mg/kg) significantly reduced the increase in in FE_{Na} (FIG. 3a) in diabetic rats at 96 hours. In addition an important and significant increase of FE_{Na} (FIG. 3b) was observed in diabetic at 96 hours after the administration of the contrast agent. Metalloporphyrin A (at 30-1000 μg/kg) produced significant reduction in a dose dependent manner in FE_{Na} (FIG. 3b) at 96 hours. Similarly, the treatment with NAC (10 mg/kg) produced significant reductions in FE_{Na} at 96 hours.

### C. Effects of Metalloporphyrin A on Kidney MPO Activity and MDA Levels

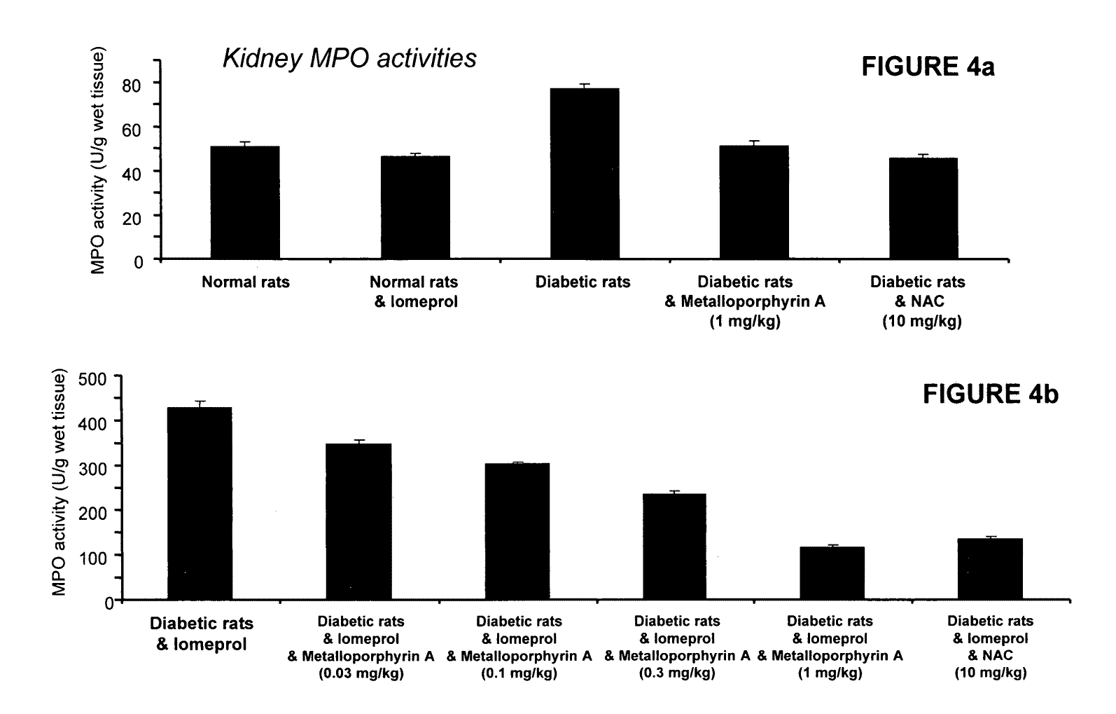

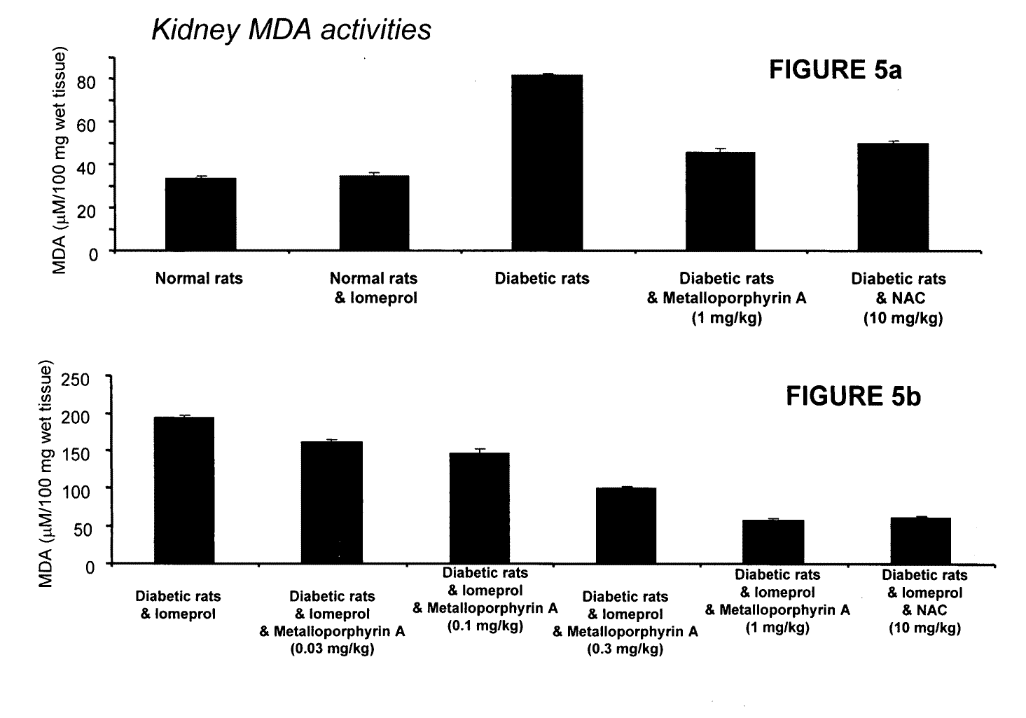

A significant increase in MPO activity (FIG. 4a) and MDA levels in the kidney (FIG. 5a) was observed in diabetic rats at 96 hours after the administration of saline in comparison to non diabetic animals (FIG. 4a). Metalloporphyrin A (at the dose of 1000 μg/kg) produced significant reductions in MPO activity (FIG. 4a) and MDA levels in the kidney (FIG. 5a) at 96 hours in diabetic animals. Similarly, the treatment with NAC (10 mg/kg) significantly reduced the increase in increase in MPO activity (FIG. 4a) and MDA levels in the kidney (FIG. 5a) in diabetic rats at 96 hours. In addition, an important and significant increase of MPO activity (FIG. 4b) and MDA levels in the kidney (FIG. 5b) was observed in diabetic at 96 hours after the administration of the contrast agent. Metalloporphyrin A (at 30-1000 μg/kg) produced significant reduction in a dose dependent manner in MPO activity (FIG. 4b) and MDA levels in the kidney (FIG. 5b) at 96 hours. Similarly, the treatment with NAC (10 mg/kg) produced significant reductions in MPO activity (FIG. 4b) and MDA levels in the kidney (FIG. 5b) at 96 hours.

### D. Effects of Metalloporphyrin A on CIN-Mediated Renal Histopathology

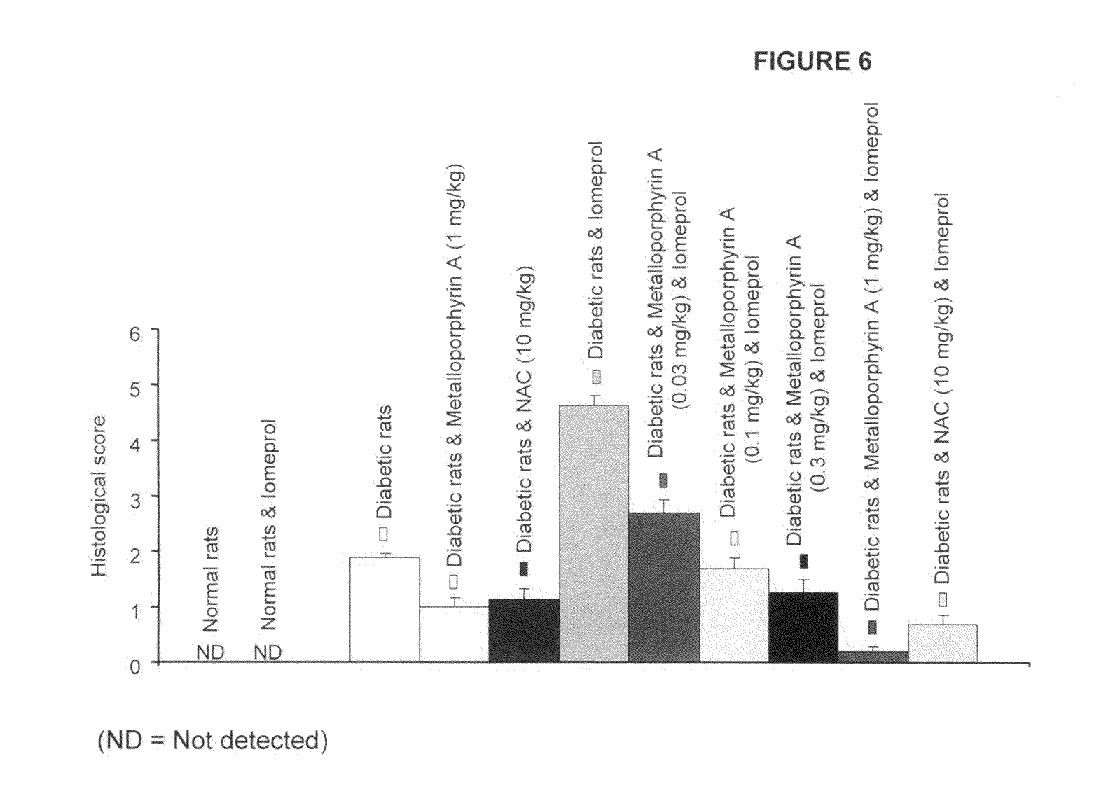

No histological alterations in the outer medulla were observed in the kidney section from Wild-type rats+placebo+saline (see histological score FIG. 6) as well as from Wild-type rats+placebo+Iomeprol sham-operated rats (histological score FIG. 6). A moderate kidney injury was observed in the kidney from diabetic rats at 96 hours after the administration of saline (see histological score FIG. 6). Metalloporphyrin A (at the dose of 1000 μg/kg) produced significant reductions of kidney injury (see histological score FIG. 6) at 96 hours in diabetic animals. Similarly, the treatment with NAC (10 mg/kg) significantly reduced the kidney damage (see histological score FIG. 6) in diabetic rats at 96 hours. In addition, a severe kidney injury was observed in diabetic at 96 hours after the administration of the contrast agent (see histological score FIG. 6). Metalloporphyrin A (at 30-1000 μg/kg) produced significant reduction of kidney injury (see histological score FIG. 6) at 96 hours. Similarly, the treatment with NAC (10 mg/kg) produced significant reductions of kidney injury (see histological score FIG. 6) at 96 hours.

### E. Plasma NGAL

Neutrophil gelatinase-associated lipocalin (NGAL) has recently been proposed as a real-time indicator of active kidney damage by Mori, K., Nakao, K., Kidney International (2007) 71, 967-970. The plasma from 5 different diabetic animals per experimental group were taken for measurement of NGAL by ELISA as outlined below:

1) For diabetic rats without contrast agent, non-diluted plasma was used. For diabetic rats with contrast agent, plasma samples were diluted 1:400 with saline prior to use.

100 microliter diluted/non-diluted plasma samples or recombinant NGAL protein (25-1000 ng/ml) were added to pre-coated ELISA plates containing a monoclonal antibody raised against NGAL. Incubation with the samples was allowed to proceed for 60 min at room temperature on a rotating platform.

2) Following incubation, plates were washed with a wash-buffer and each well thereafter incubated with 100 microliters of a biotinylated anti-NGAL monoclonal antibody for 60 minutes at room temperature on a rotating platform.

3) Following washing, wells were incubated with 100 microliters of a HRP-streptavidin conjugate for 60 minutes on a rotating platform.

4) Following washing, 100 microliters of TMB substrate was added to each well and color allowed to develop for 15 min prior to the addition of stop solution.

5) Plates were read at an absorbance of 450 nm.

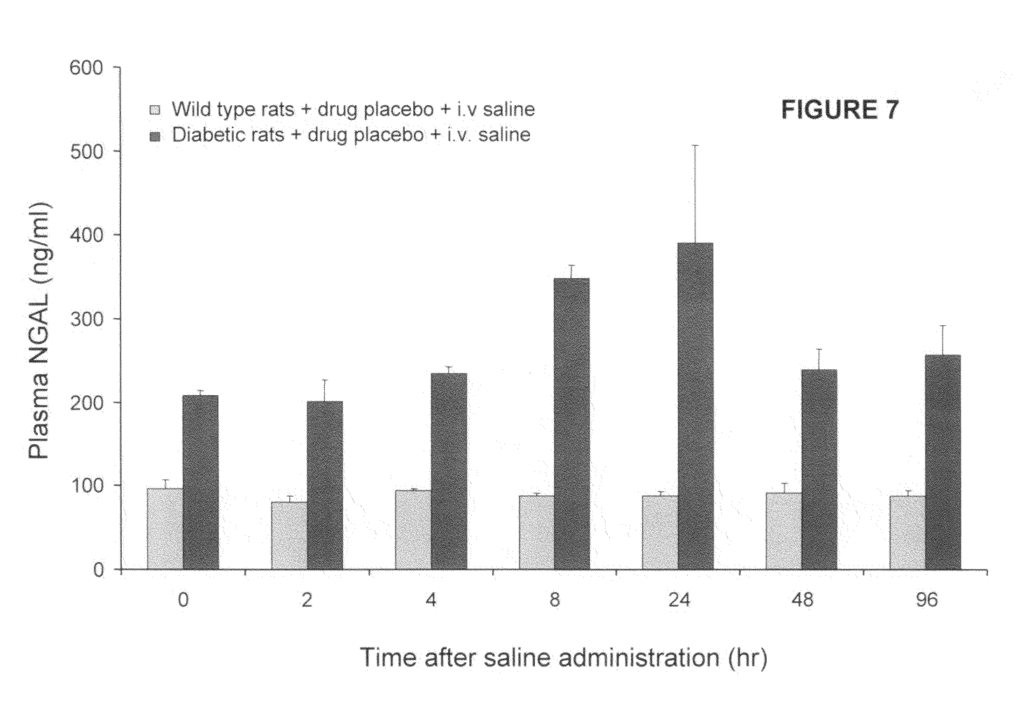

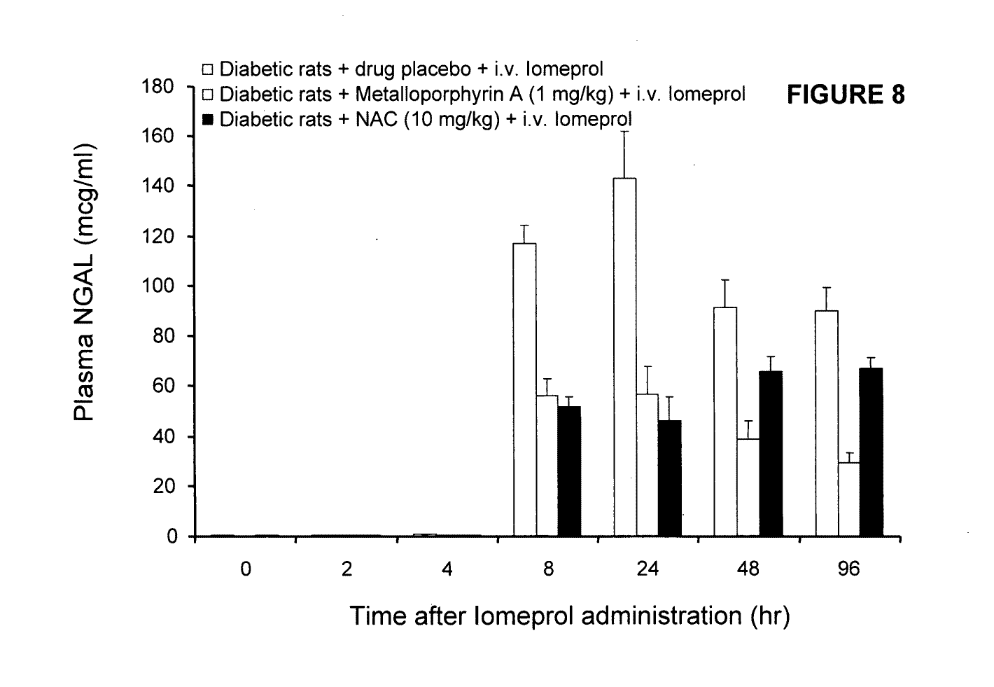

The results of the NGAL studies are shown in FIGS. 7 and 8. FIG. 7 shows the results of the plasma NGAL (ng/ml) in Wild type rats relative to the diabetic rats in the control groups administered a drug placebo and i.v.saline at 0, 2, 4, 8, 24, 48 and 96 hours. FIG. 8 shows the results of the plasma NGAL (ng/ml) in three groups of diabetic rats. The first group were administered a drug placebo and i.v.contrast agent (Iomeprol), the second group were administered Metalloporphyrin A (1 mg/kg) and i.v.contrast agent (Iomeprol) and the third group were administered NAC (10 mg/kg) and i.v.contrast agent (Iomeprol). The plasma NGAL readings were taken at 0, 2, 4, 8, 24, 48 and 96 hours. FIG. 8 shows that plasma NGAL levels for the groups of diabetic rats administered Metalloporphyrin A and NAC are significantly lower than the group of rats administered a drug placebo.

### F. Effects of Metalloporphyrin A on ICAM-1, Nitrotyrosine and Par Expression.

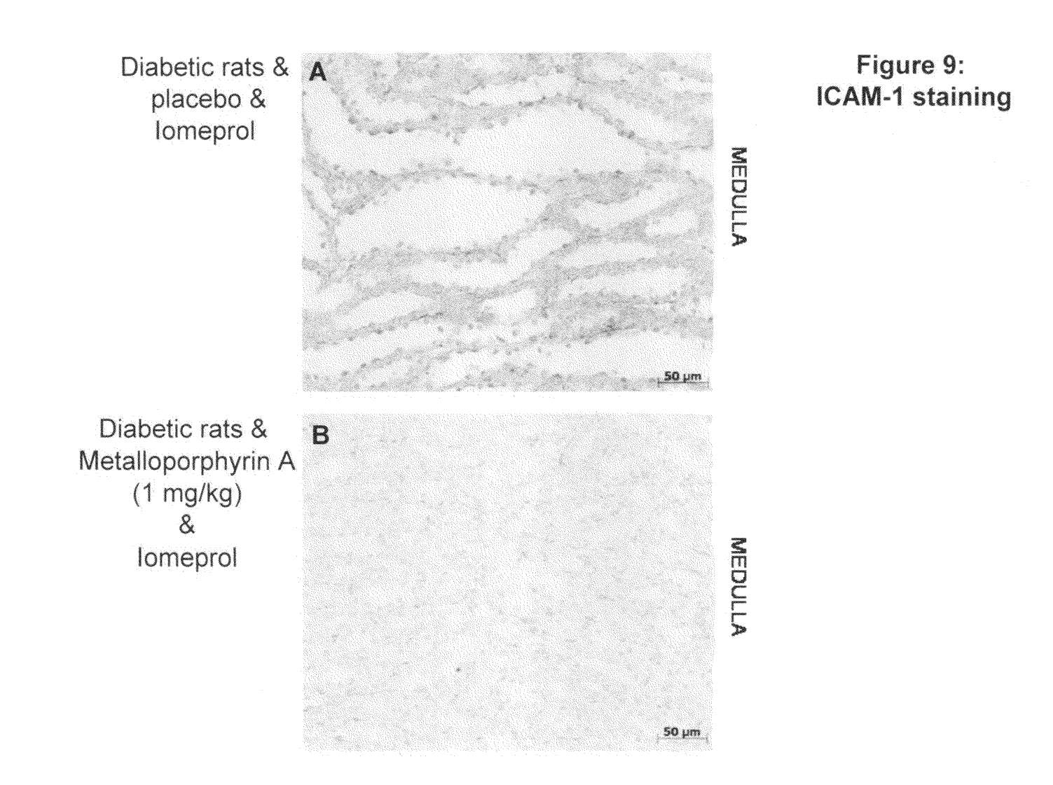

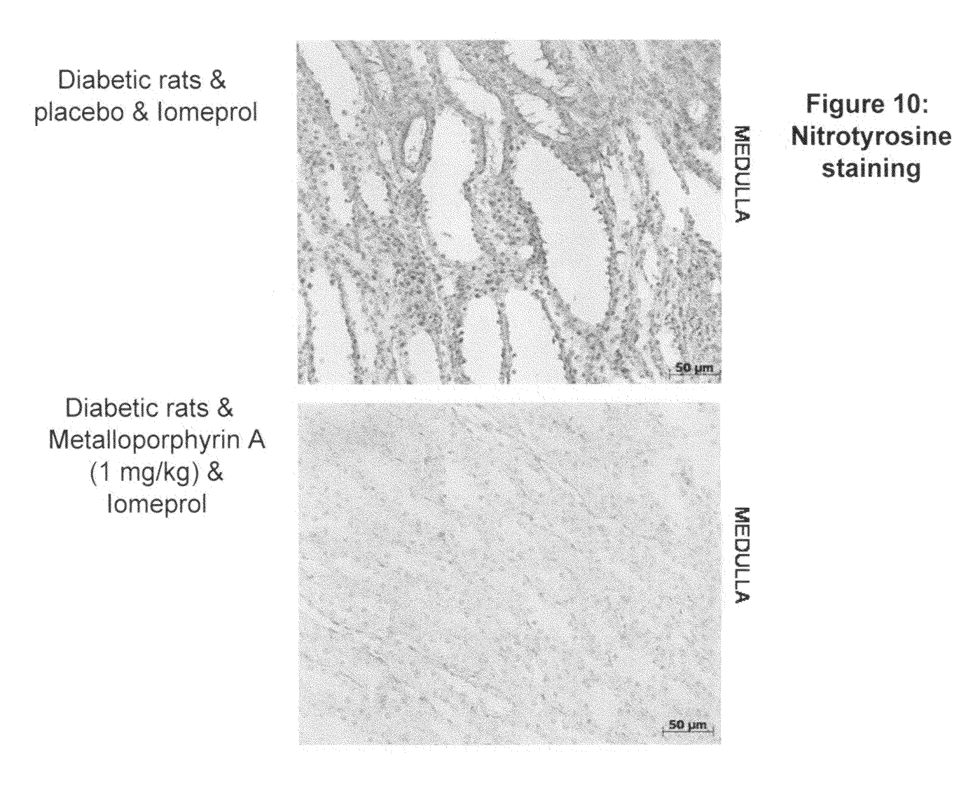

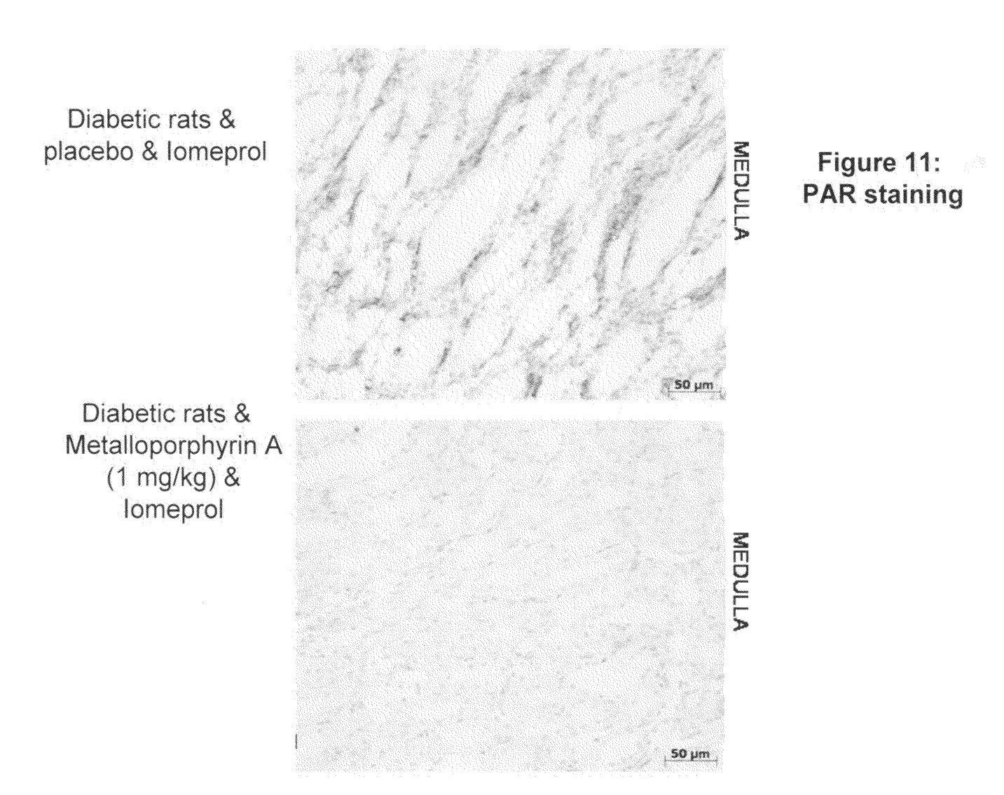

To further elucidate the effect of Metalloporphyrin A on kidney injury in diabetic rats, the expression of ICAM-1 (FIG. 9), Nitrotyrosine (FIG. 10) and PAR (FIG. 11) in response to Iomeprol administration were screened using specific monoclonal antibodies. There was no evidence of staining for the 3 different inflammatory markers from wild-type rats in the presence or absence of Iomeprol. Interestingly, moderate positive staining was seen for all markers from diabetic rats only which was attenuated by Metalloporphyrin A. As anticipated, administration of Iomeprol to diabetic rats caused significant staining of ICAM-1 (FIG. 9A), Nitrotyrosine (FIG. 10A) and PAR (FIG. 11A). Additionally evident is the loss in general integrity of the medulla regions typified by swelling, vacuoles and loss of basement membrane architecture. Administration of metalloporphyrin A (1 mg/kg) 60 min prior to Iomeprol administration significantly reduced the staining of all 3 inflammatory markers (FIGS. 9B, 10B and 11B) and also improved structural integrity of the medulla. Similarly to metalloporphyrin A, treatment with NAC (10 mg/kg) also afforded protection against ICAM-1, Nitrotyrosine and PAR.

### Example 2

### Effects of N-Acetyl Cysteine (NAC) and Metalloporphyrin A on Contrast-Induced Nephropathy in Diabetic Rats

Male Wistar rats (150-200 g; Harlan Nossanr) were housed in a controlled environment and provided with standard rodent chow and water. Diabetes was induced after 2 hours of fasting. The animals received a single 60 mg/kg intravenous (i.v.) injection of streptozotocin (Sigma, St. Louis, Mo.) in 10 mM sodium citrate buffer, pH 4.5 (see figure). Control non-diabetic animals were fasted and received citrate buffer alone. After 24 hours, animals with blood glucose levels greater than 250 mg/dl were considered diabetic. The diabetic state was confirmed by evaluating the blood glucose levels. Ten days following the induction of diabetes rats were anesthetized with 90 mg/kg ketamine i.m. and 10 mg/kg xylazine i.m. and were treated with the contrast agent iomeprol (10 mL/kg injected via the lateral tail vein) or with 0.9% normal saline. The contrast agent used was the low osmolar non-ionic monomer iomeprol (Iomeron, 400 mgI mL-1; Bracco SpA, Milan, Italy) with an osmolality of 726±34 mosmol/kg H₂O.

The animals were then randomly allocated into groups as described below:

| Groups | Treatments |
|---|---|
| 1 | STZ + Vehicle |
| 2 | STZ + Iomeperol |
| 3 | STZ + Iomeprol + NAC (3 mg/kg, i.v.) given at −30 min. |
| 4 | STZ + Iomeprol + NAC (10 mg/kg, i.v.) given at −30 min. |
| 5 | STZ + Iomeprol + NAC (30 mg/kg, i.v.) given at −30 min. |
| 6 | STZ + Iomeprol + NAC (100 mg/kg, i.v.) given at −30 min. |
| 7 | STZ + Iomeprol + NAC (10 mg/kg, i.v.) given at −30 min. + |
| | Metalloporphyrin A (1 mg/kg, i.v.) given at −30 min. |
| 8 | STZ + Iomeprol + NAC (30 mg/kg, i.v.) given at −30 min. + |
| | Metalloporphyrin A (1 mg/kg, i.v.) given at −30 min. |
| 9 | STZ + Iomeprol + NAC (100 mg/kg, i.v.) given at −30 min. + |
| | Metalloporphyrin A (1 mg/kg, i.v.) given at −30 min. |

Blood samples were collected via the lateral tail vein into S1/3 tubes containing serum gel at 0 and 24 hours. The samples were centrifuged (6000 r.p.m. for 3 min) to separate plasma. All plasma samples were analyzed for biochemical parameters within 24 hours after collection. Plasma and urine concentrations of creatinine were measured as indicators of impaired glomerular function. Creatinine clearance was calculated using the following formula (=UV/P), where U refers to Creatinine concentration in urine, V to urine volume/min and P to serum creatinine.

### Measurement of Protein Concentration.

Protein concentration in urine was determined by Bio-Rad DC Protein Assay (BioRad, Richmond Calif.). The Bio-Rad DC protein assay is a colorimetric assay for protein concentration. The reaction is similar to the well-documented Lowry assay (Lowry et al., Protein measurement with the Folin phenol reagent, J Biol Chem 193, 265-275 (1951). The Bio-Rad DC protein assay requires only a single 15-minute incubation, and absorbance is stable for a least 2 hours. The amount of protein is expressed in mg/ml.

### Results

### Effect of Metalloporphyrin A and NAC on CIN-Mediated Glomerular Dysfunction

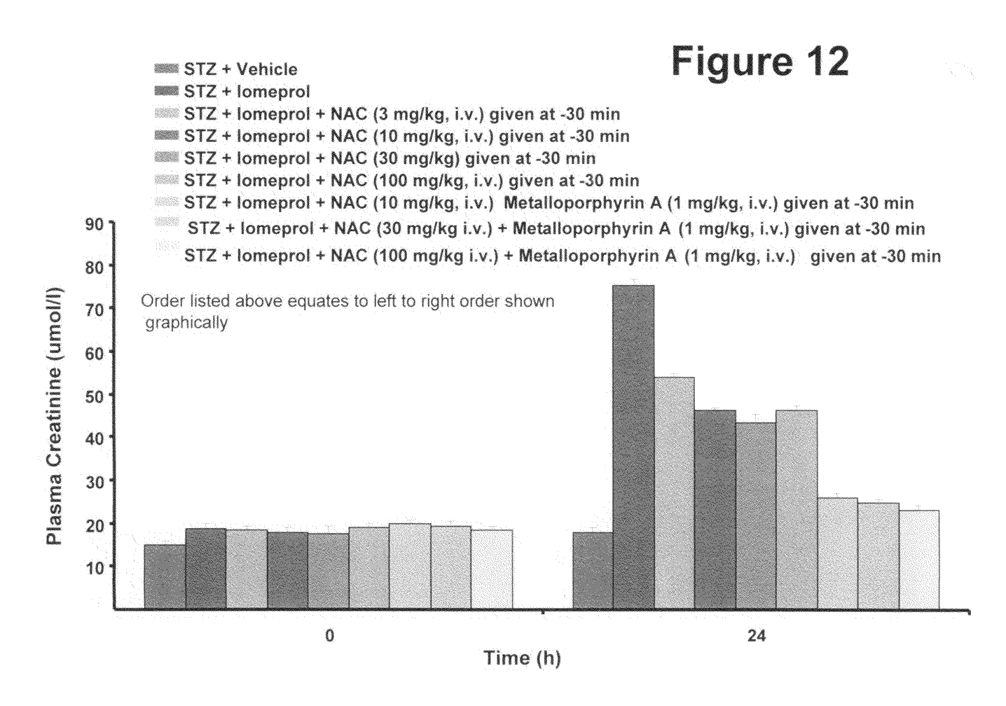

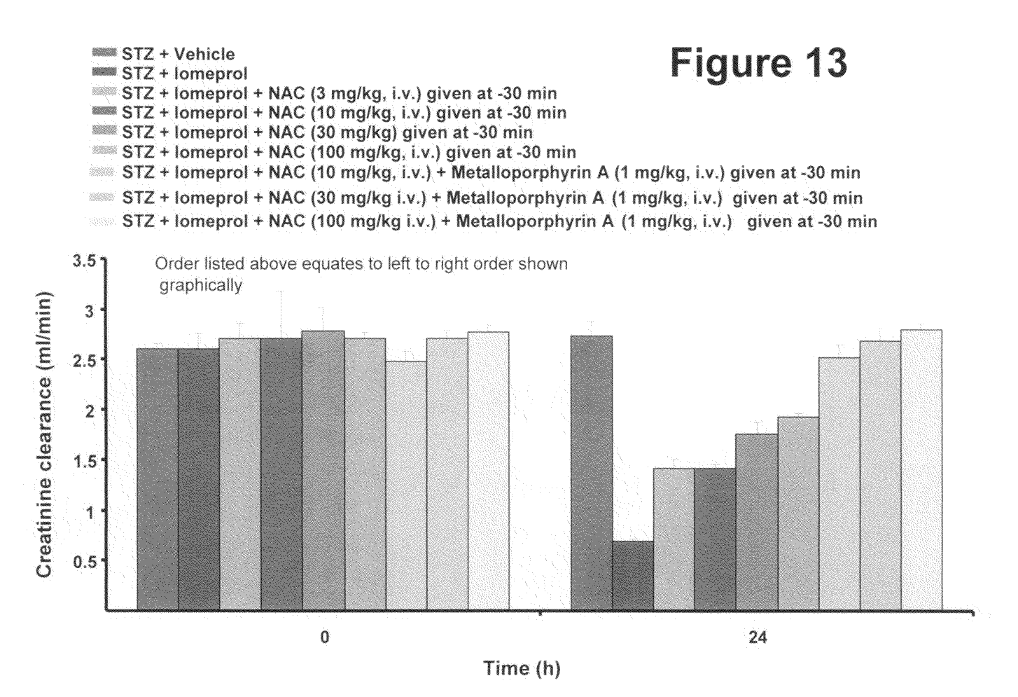

A significant increase of the plasma concentrations of creatinine (FIG. 12) as well as a significantly lower creatinine clearance (FIG. 13) was observed in diabetic rats at 24 h after the administration of the contrast agent. The treatment with NAC and Metalloporphyrin A+NAC produced significant reductions of plasma creatinine (FIG. 12) as well as a significant increase in creatinine clearance (FIG. 13).

### Effect of Metalloporphyrin A and NAC on Urine αGST Levels.

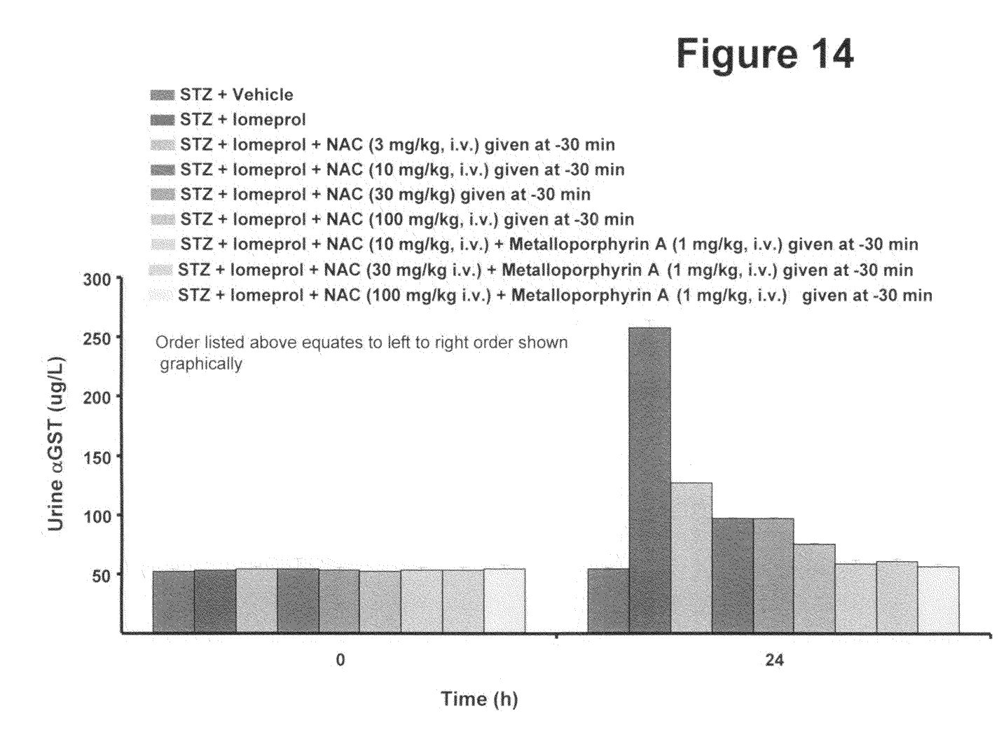

A significant increase in the urine concentrations of αGST (FIG. 14) was observed in diabetic rats at 24 hours after the administration of the contrast agent. The treatment with NAC as well as with Metalloporphyrin A+NAC produced a significant reduction in urine αGST (FIG. 14) concentrations at 24 hours after the administration of the contrast agent.

### Effect of Metalloporphyrin A and NAC on Urine Protein Concentration.

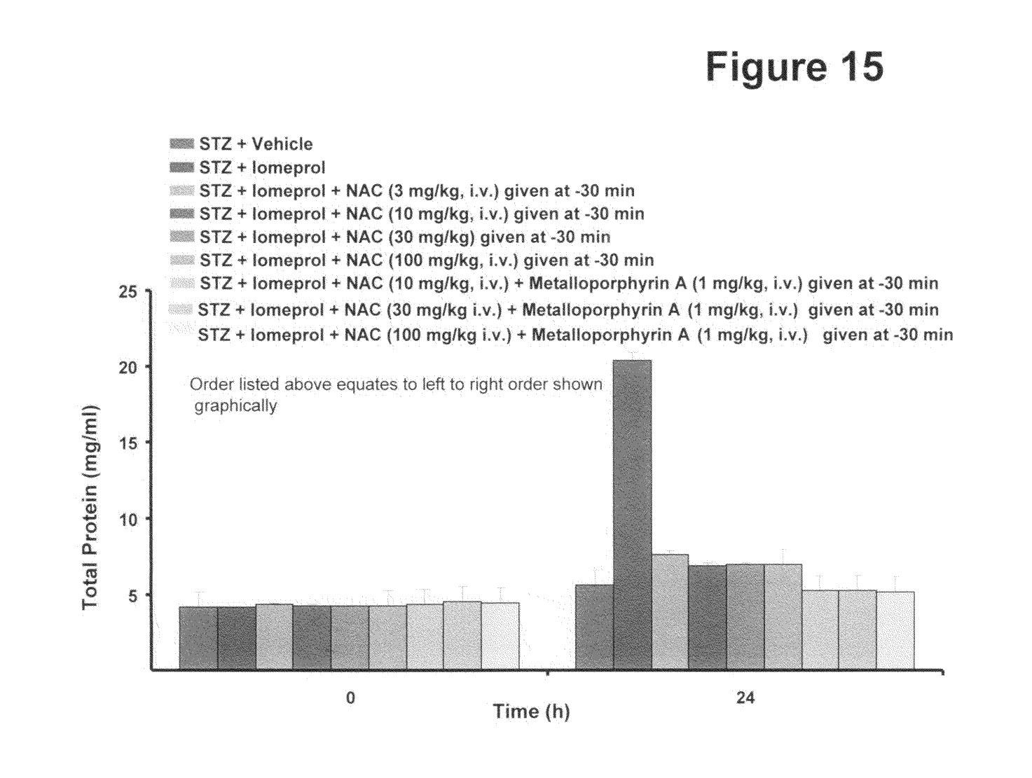

An increase of the urine concentrations of total protein (FIG. 15) was observed in diabetic rats at 24 hours after the administration of the contrast agent. The treatment with NAC as well as with Metalloporphyrin A+NAC produced significant reductions of total protein concentration in the urine (FIG. 15).

### Example 3

### Effects of Super Oxide Dismutase Mimic (SODm) M40403 Alone and in Combination with N-Acetyl Cysteine (NAC) on Contrast-Induced Nephropathy in Diabetic Rats

Male Wistar rats (150-200 g; Harlan Nossanr) were housed in a controlled environment and provided with standard rodent chow and water. Diabetes was induced after 12 hours of fasting. The animals received a single 60 mg/kg intravenous (i.v.) injection of streptozotocin (Sigma, St. Louis, Mo.) in 10 mM sodium citrate buffer, pH 4.5 (see figure). Control non-diabetic animals were fasted and received citrate buffer alone. After 24 hours, animals with blood glucose levels greater than 250 mg/dl were considered diabetic. The diabetic state was confirmed by evaluating the blood glucose levels. Ten days following the induction of diabetes rats were anesthetized with 90 mg/kg ketamine i.m. and 10 mg/kg xylazine i.m. and were treated with the contrast agent iomeprol (10 mL/kg injected via the lateral tail vein) or with 0.9% normal saline. The contrast agent used was the low osmolar non-ionic monomer iomeprol (Iomeron, 400 mgI mL-1; Bracco SpA, Milan, Italy) with an osmolality of 726±34 mosmol/kg H₂O.

The animals were then randomly allocated into groups as described below

| Groups | Treatments |
|---|---|
| 1 | Diabetic rats + i.v. buffer of M40403 (ie placebo) |
| 2 | Diabetic rats + Iomeperol + i.v. buffer of M40403 |
| | given at −30 min |
| 3 | Diabetic rats + Iomeperol + M40403 (0.5 mg/kg, i.v.) |
| | given at −30 min |
| 4 | Diabetic rats + Iomeperol + NAC (3 mg/kg, i.v.) given |
| | at −30 min |
| 5 | Diabetic rats + Iomeperol + M40403 (0.5 mg/kg, i.v.) + |
| | NAC (3 mg/kg, i.v.) given at −30 min |

Blood samples were collected via the lateral tail vein into S1/3 tubes containing serum gel at 0 and 24 hours. The samples were centrifuged (6000 r.p.m. for 3 min) to separate plasma. All plasma samples were analyzed for biochemical parameters within 24 hours after collection. Plasma and urine concentrations of creatinine were measured as indicators of impaired glomerular function. Creatinine clearance was calculated using the following formula (=UV/P), where U refers to Creatinine concentration in urine, V to urine volume/min and P to serum creatinine. Plasma and urine concentrations of NGAL levels were evaluated.

### Results

### Effect of M40403, and M40403 and NAC on CIN-Mediated Glomerular Dysfunction

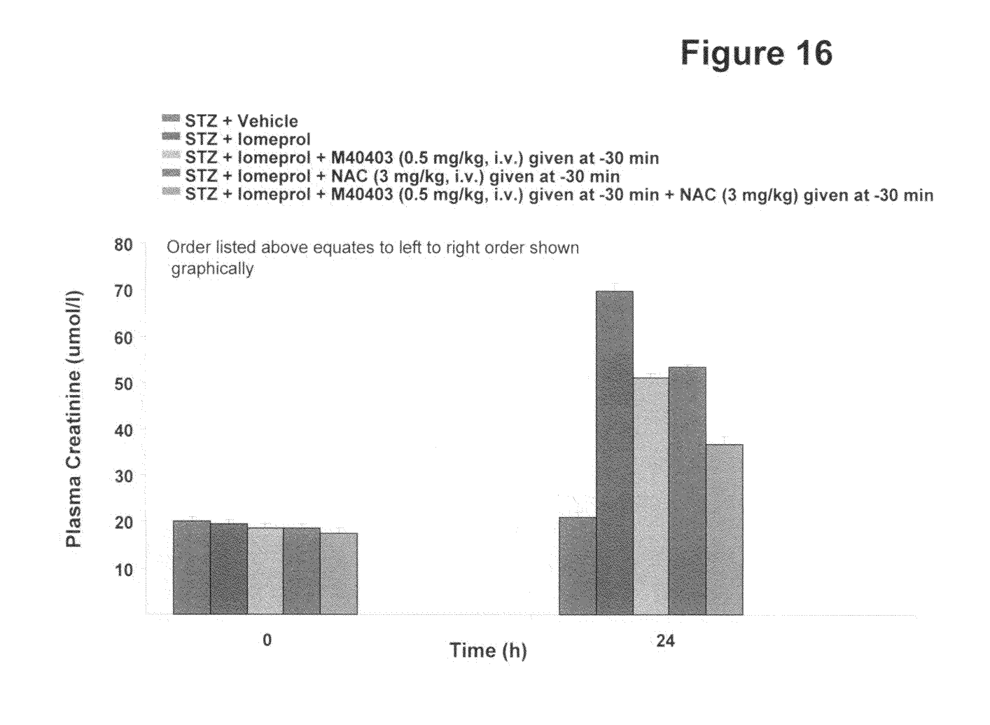

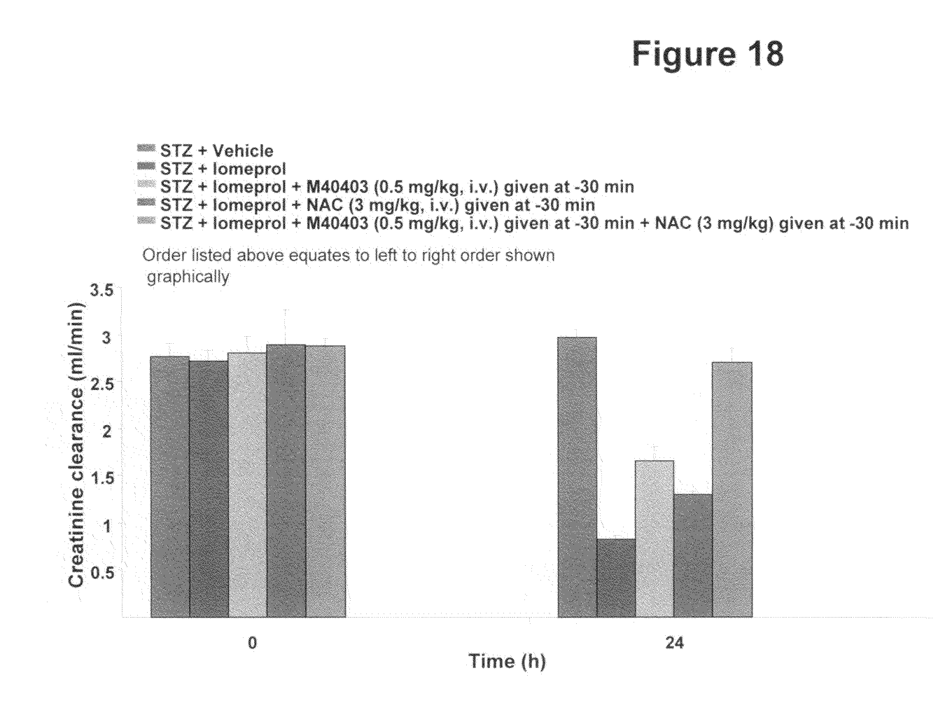

A significant increase of the plasma concentrations of creatinine (FIG. 16) as well as a significantly lower creatinine clearance (FIG. 18) was observed in diabetic rats at 24 hours after the administration of the contrast agent. The treatment with M40403, and M40403+NAC produced significant reductions of plasma creatinine (FIG. 16) as well as significantly increased creatinine clearance (FIG. 18).

### Effect of M40403, and M40403 and NAC on CIN on Na+ and K+ in Plasma Levels at 24 h After CIN Induction.

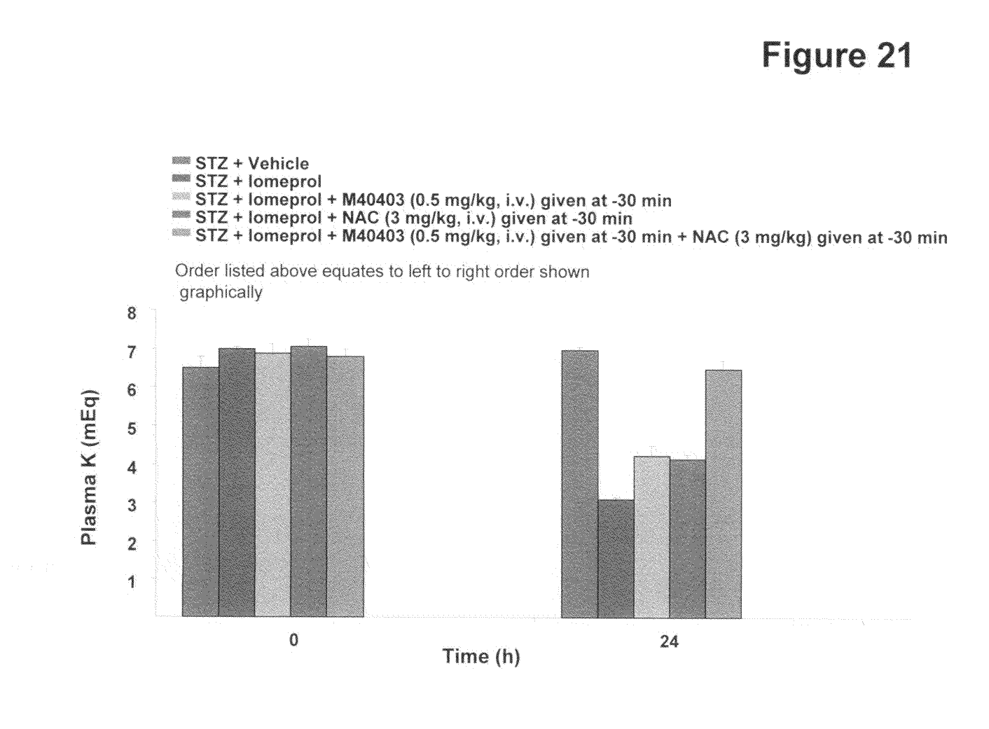

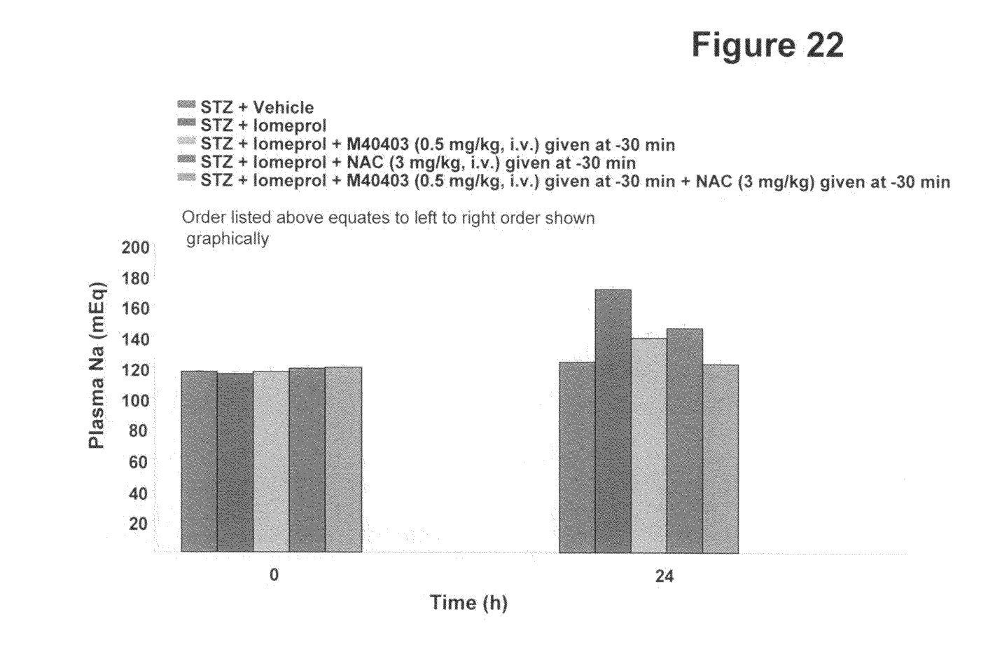

A significant increase of the plasma concentrations of Na+ (FIG. 22) as well as a significant decrease in plasma concentrations of K+ (FIG. 21) was observed in diabetic rats at 24 hours after the administration of the contrast agent. The treatment with M40403, and M40403+NAC produced significant reductions of plasma Na+ (FIG. 22) as well as a significant increase in plasma concentrations of K+ (FIG. 21).

### Effect of M40403, M40403 and NAC on Plasma and Urine NGAL Levels.

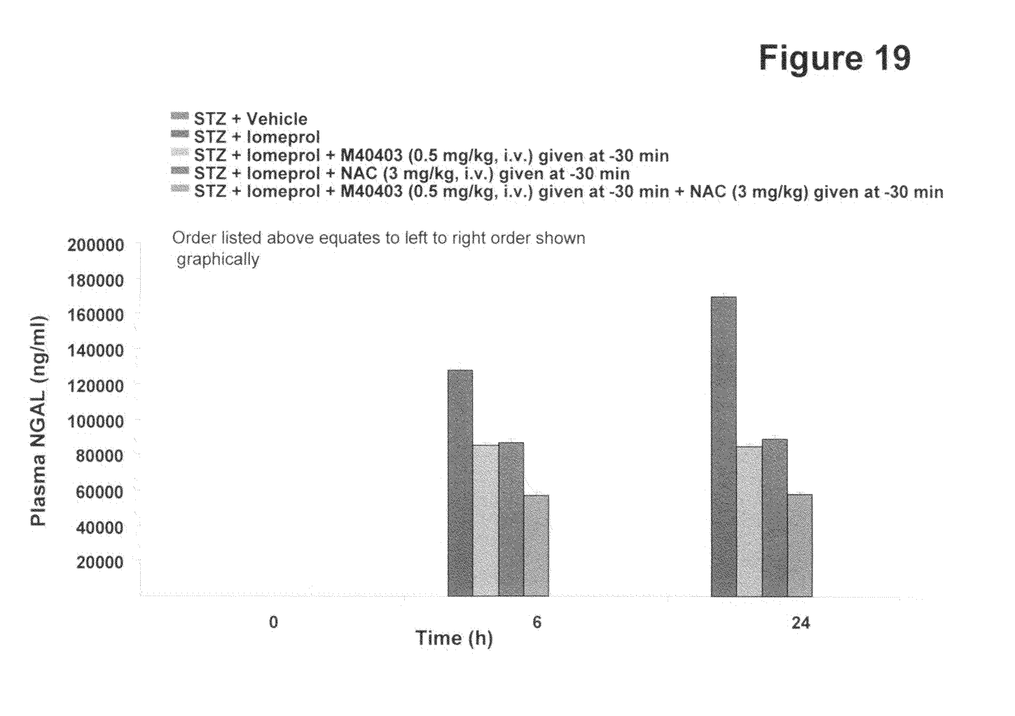

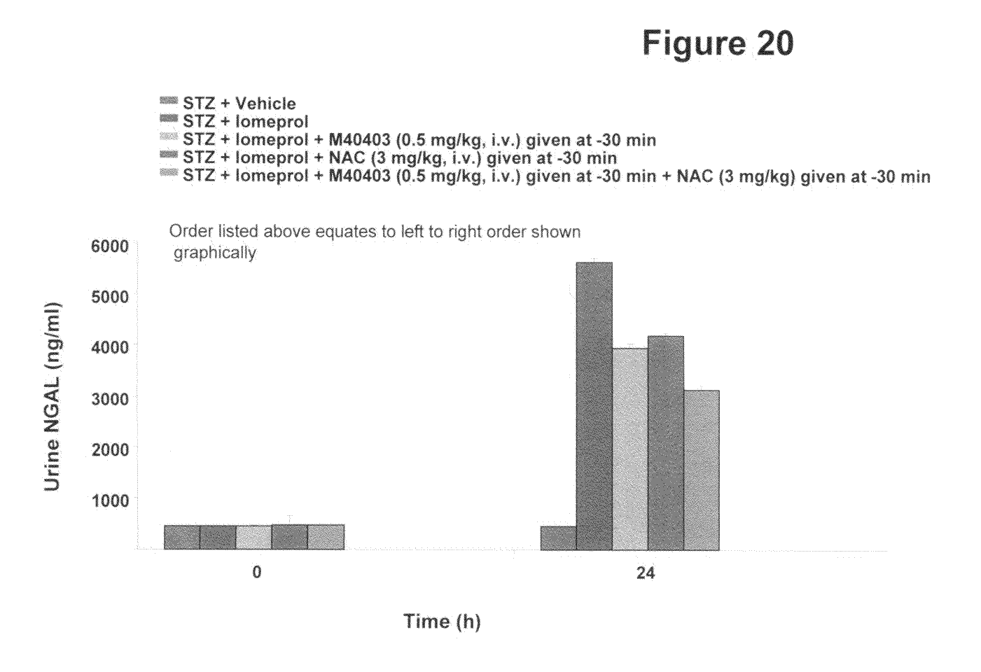

A significant increase of the plasma concentrations of NGAL (FIG. 19) was observed in diabetic rats at 24 hours after the administration of the contrast agent. The treatment with M40403, and M40403+NAC produced a significant reduction in plasma NGAL (FIG. 19) concentrations at 24 hours after the administration of the contrast agent. In addition, the NGAL levels were also evaluated in the urine samples. A significant increase in the urine concentrations of NGAL (FIG. 20) was observed in diabetic rats at 24 hours after the administration of contrast agent. The treatment with M40403, and M40403+NAC produced a significant reduction of urine NGAL (FIG. 20) concentrations at 24 hours after the administration of the contrast agent.

### Effect of M40403, M40403 and NAC on Urine Protein Concentration.

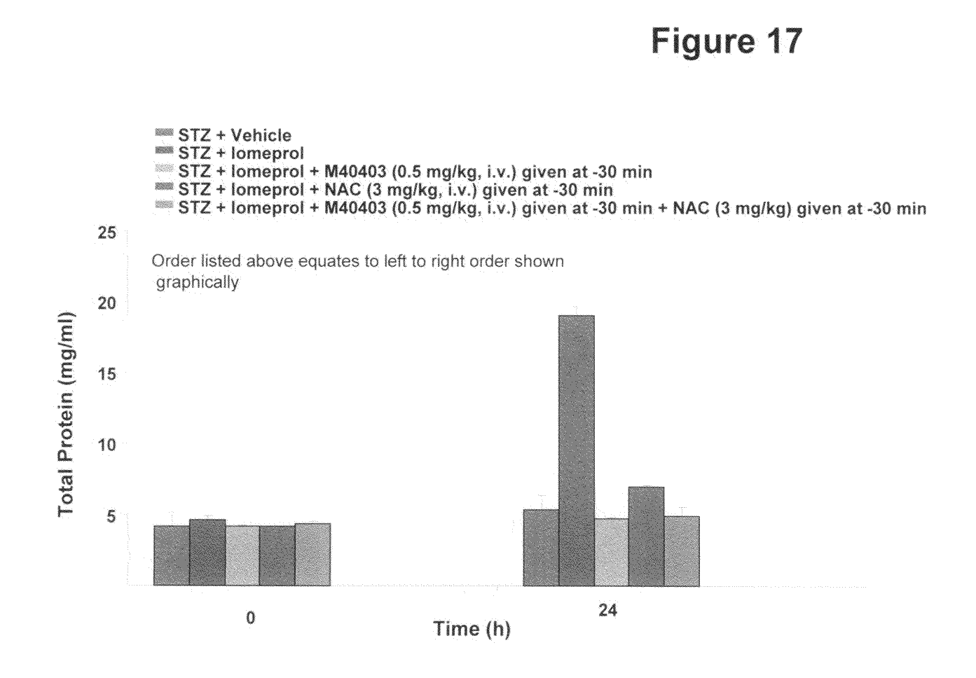

A significant increase of the urine concentrations of total protein (FIG. 17) was observed in diabetic rats at 24 hours after the administration of the contrast agent. The treatment with M40403, and M40403+NAC produced significant reductions of plasma creatinine (FIG. 16) as well as a significantly reduced the total protein concentrations in the urine (FIG. 17).

### Example 4

### Effect of PARP Inhibitors on the Prophylaxis of Contrast Induced Nephropathy

Male Wistar rats (150-200 g; Harlan Nossanr) were housed in a controlled environment and provided with standard rodent chow and water. Diabetes was induced after 12 hours of fasting. The animals received a single 60 mg/kg intravenous (i.v.) injection of streptozotocin (Sigma, St. Louis, Mo.) in 10 mM sodium citrate buffer, pH 4.5 (see figure). Control non-diabetic animals were fasted and received citrate buffer alone. After 24 hours, animals with blood glucose levels greater than 250 mg/dl were considered diabetic. The diabetic state was confirmed by evaluating the blood glucose levels. Ten days following the induction of diabetes rats were anesthetized with 90 mg/kg ketamine i.m. and 10 mg/kg xylazine i.m. and were treated with the contrast agent iomeprol (10 mL/kg injected via the lateral tail vein) or with 0.9% normal saline. The contrast agent used was the low osmolar non-ionic monomer iomeprol (Iomeron, 400 mgI mL-1; Bracco SpA, Milan, Italy) with an osmolality of 726±34 mosmol/kg H₂O.

The animals were randomly allocated to different groups. Contrast agent and one of three PARP inhibitors were administered as shown in the following scheme:

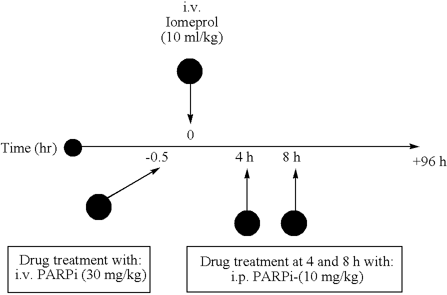

The three PARP inhibitors have the following structure and activity:

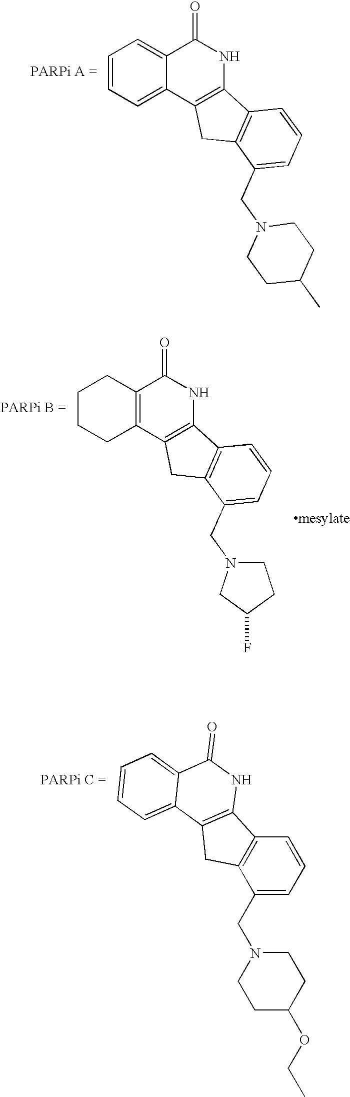

### Measurement of Biochemical Parameters

At the indicated time point blood samples were collected via the lateral tail vein into S1/3 tubes containing serum gel at intervals out to 96 hours. The samples were centrifuged (6000 r.p.m. for 3 min) to separate plasma. All plasma samples were analyzed for biochemical parameters within 24 hours after collection. Plasma urea, plasma and urine concentrations of creatinine were measured as indicators of impaired glomerular function. Plasma concentrations of NGAL (neutrophil gelatinase associated lipocalin) levels and urine αGST (alpha glutathione S-transferase) and NGAL levels were evaluated.

### Histologic Evaluation

At postmortem, a 5 μm section of kidney was removed and placed in formalin and processed through to wax. Five millimeter sections were cut and stained with hematoxylin and eosin. Histologic assessment of outer medulla damage was examined by an experienced morphologist, who was not aware of the sample identity. The criteria for injury/necrosis were the following: 0=normal histology; 1=minor edema, minor cell swelling; 2=haemorrhage, moderate edema, moderate cells vacuolization and swelling; 3=moderate haemorrhage, moderate edema, moderate cells vacuolization, swelling and chromatin alteration; 4=severe edema, severe cells vacuolization, swelling and chromatin alteration, presence of necrosis spot; 5=severe edema, severe cells vacuolization, swelling and chromatin alteration, severe necrosis.

### Results

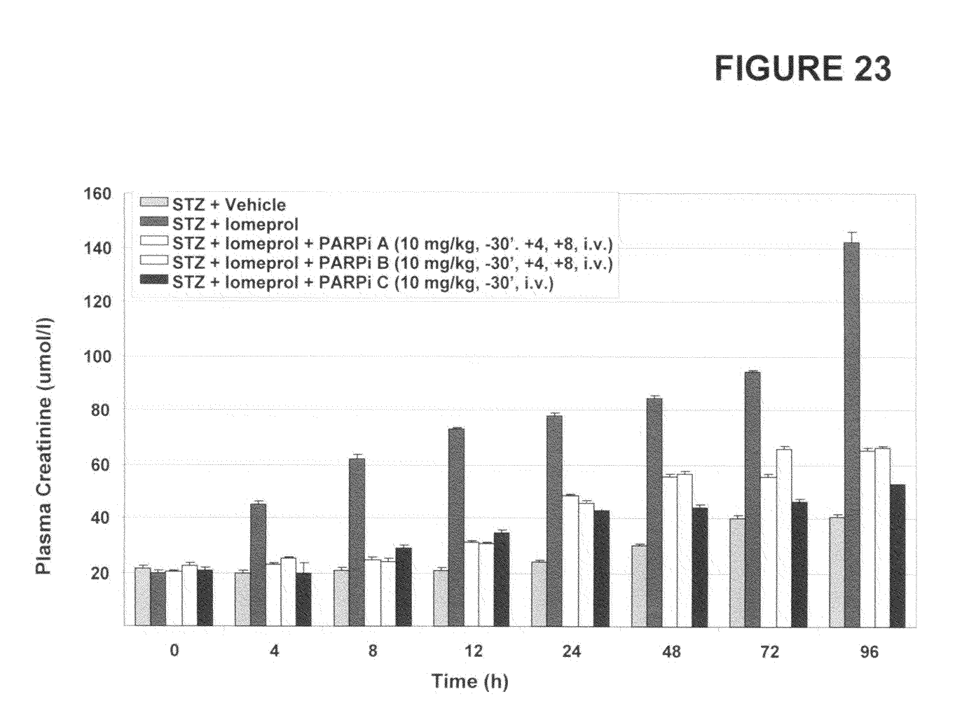

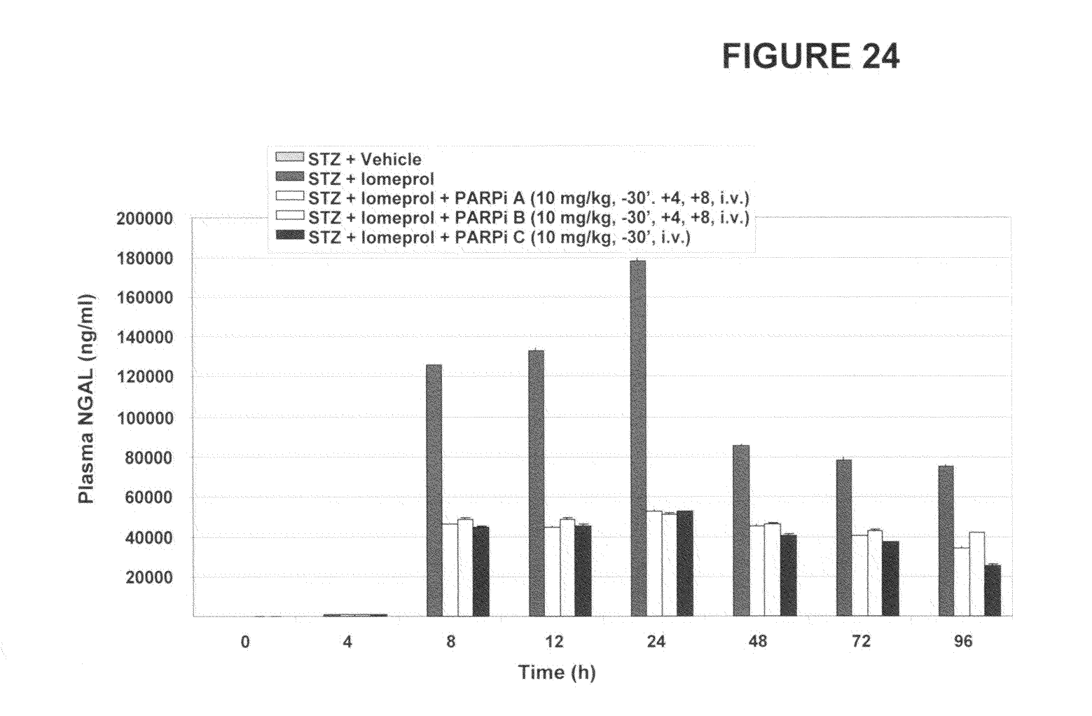

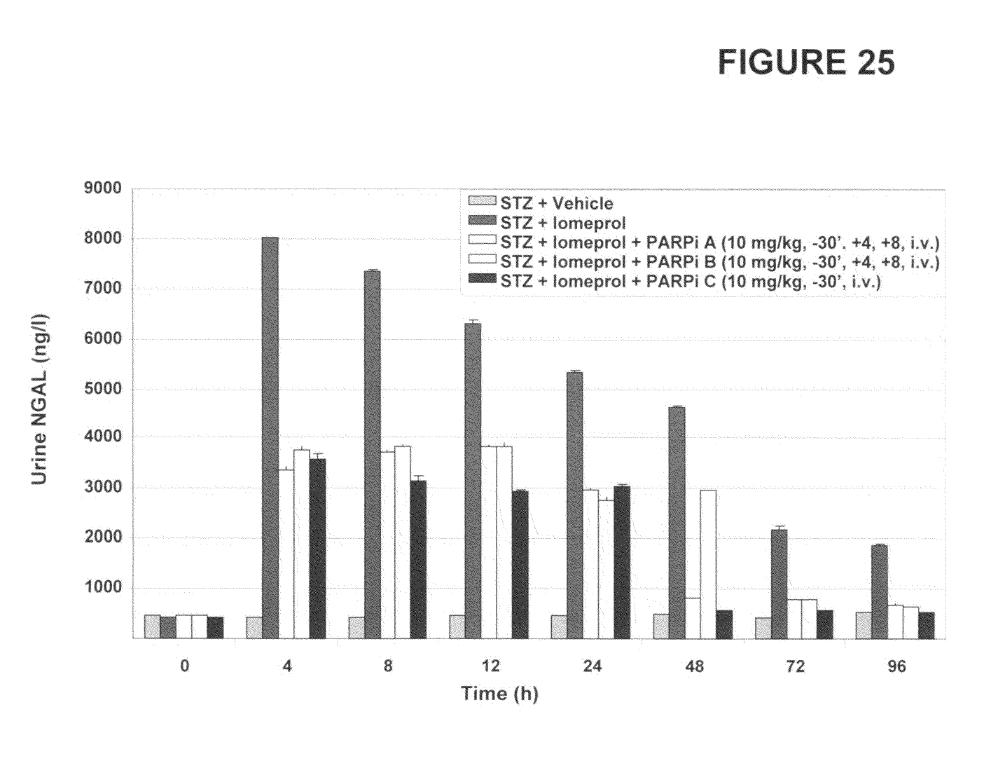

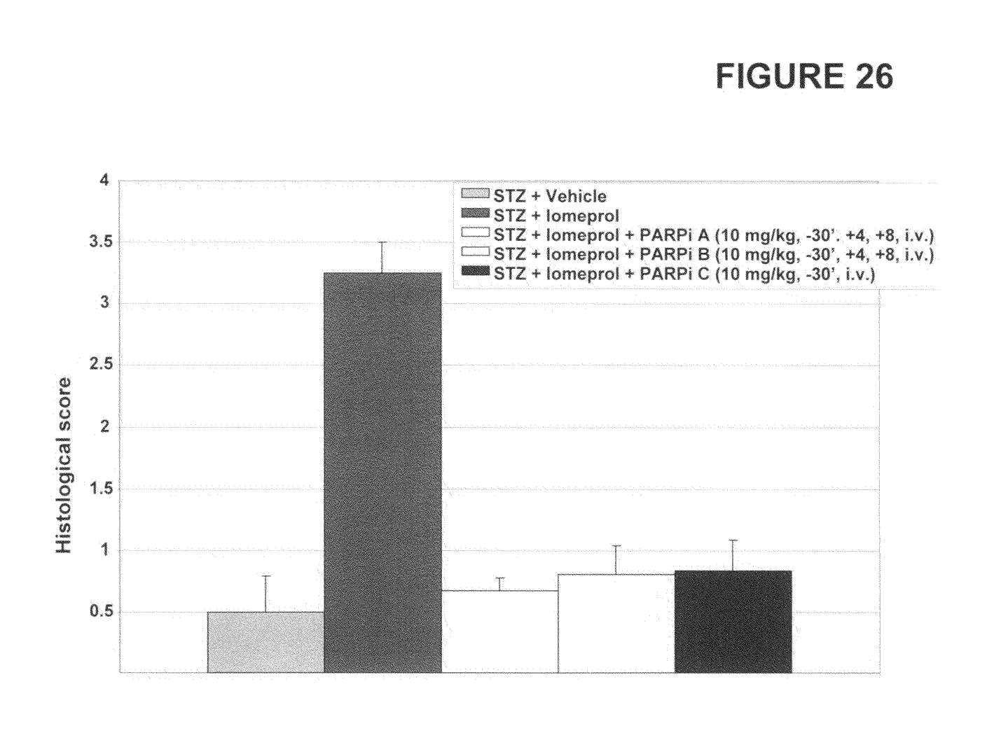

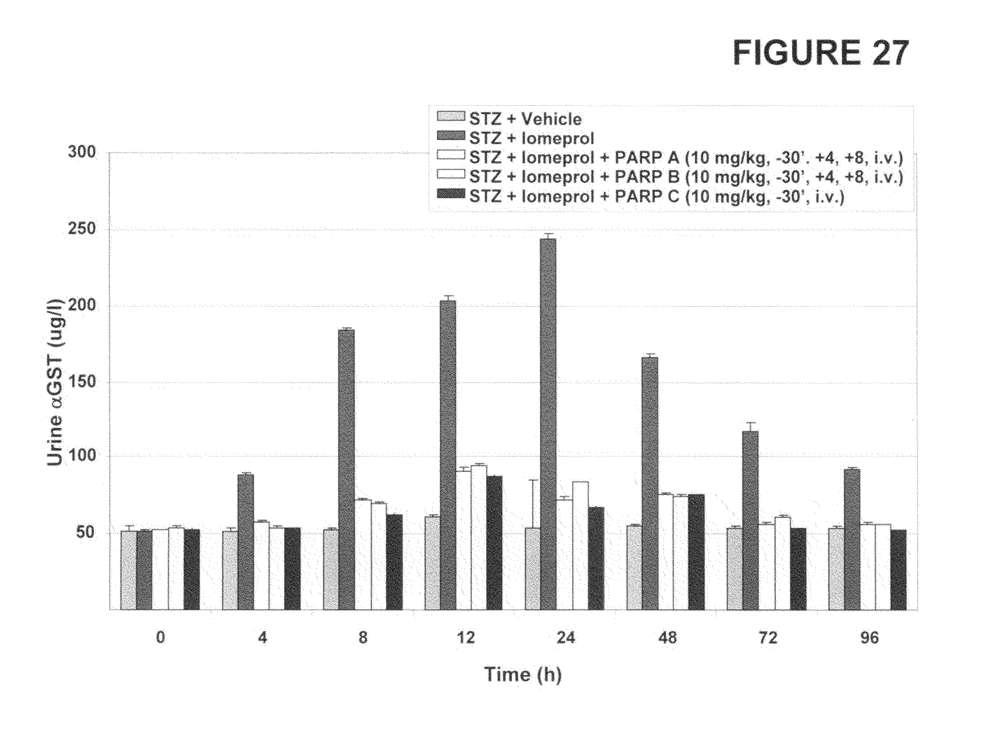

The results are shown graphically in FIGS. 23 to 27. It can be seen from the Figures that administration of a PARP inhibitor reduces the level of plasma creatinine, and NGAL, urine NGAL and αGST relative to the levels seen with contrast agent alone. The histological scores are also seen to be reduced (FIG. 26). From these results it is further expected that a combination of a PARP inhibitor with Metalloporphyrin A the peroxynitrite decomposition agent would additionally show good activity in the CIN model.

### Example 5

### Effect of PARP Inhibitor INO 1001 and INO 1001 in Combination with NAC on the Prophylaxis of Contrast Induced Nephropathy

Male Wistar rats (150-200 g; Harlan Nossanr) were housed in a controlled environment and provided with standard rodent chow and water. Diabetes was induced after 12 h of fasting. The animals received a single 60 mg/kg intravenous (i.v.) injection of streptozotocin (Sigma, St. Louis, Mo.) in 10 mM sodium citrate buffer, pH 4.5 (see figure). Control non-diabetic animals were fasted and received citrate buffer alone. After 24 hours, animals with blood glucose levels greater than 250 mg/dl were considered diabetic. The diabetic state was confirmed by evaluating the blood glucose levels. Ten days following the induction of diabetes rats were anesthetized with 90 mg/kg ketamine i.m. and 10 mg/kg xylazine i.m. and were treated with the contrast agent iomeprol (10 mL/kg injected via the lateral tail vein) or with 0.9% normal saline. The contrast agent used was the low osmolar non-ionic monomer iomeprol (Iomeron, 400 mgI mL-1; Bracco SpA, Milan, Italy) with an osmolality of 726±34 mosmol/kg H₂O.

The animals were randomly allocated into the following different groups.

GROUP 1: Diabetic rats only+Placebo (i.v.).
GROUP 2: Diabetic rats+Iomeprol+Placebo (i.v.) given at −30 min.
GROUP 3: Diabetic rats+Iomeprol+NAC (30 mg/kg, i.v.) given at −30 min.
GROUP 4: Diabetic rats+Iomeprol+INO-1001 (10 mg/kg, ip) given at −30 min.
GROUP 5: Diabetic rats+Iomeprol+NAC (30 mg/kg, i.v.)+INO-1001 (10 mg/kg, ip) given at −30 min.

### Measurement of Biochemical Parameters

Blood samples were collected via the lateral tail vein into S1/3 tubes containing serum gel at 24 hours. The samples were centrifuged (6000 r.p.m. for 3 min) to separate plasma. All plasma samples were analyzed for biochemical parameters within 24 hours after collection. Plasma and urine concentrations of creatinine were measured as indicators of impaired glomerular function. Creatinine clearance was calculated using the following formula (=UV/P), where U refers to Creatinine concentration in urine, V to urine volume/min and P to serum creatinine. Plasma and urine concentrations of NGAL (neutrophil gelatinase associated lipocalin) levels and urine αGST (alpha glutathione S-transferase) and NGAL levels were evaluated.

### Measurement of Protein Concentration.

Protein concentration in urine was determined by Bio-Rad DC Protein Assay (BioRad, Richmond Calif.). The Bio-Rad DC protein assay is a colorimetric assay for protein concentration. The reaction is similar to the well-documented Lowry assay (Lowry et al., Protein measurement with the Folin phenol reagent, J Biol Chem 193, 265-275 (1951). The Bio-Rad DC protein assay requires only a single 15-minute incubation, and absorbance is stable for a least 2 hours. The amount of protein is expressed in mg/ml.

### Results

### Effect of INO-1001 and NAC on CIN-Mediated Glomerular Dysfunction

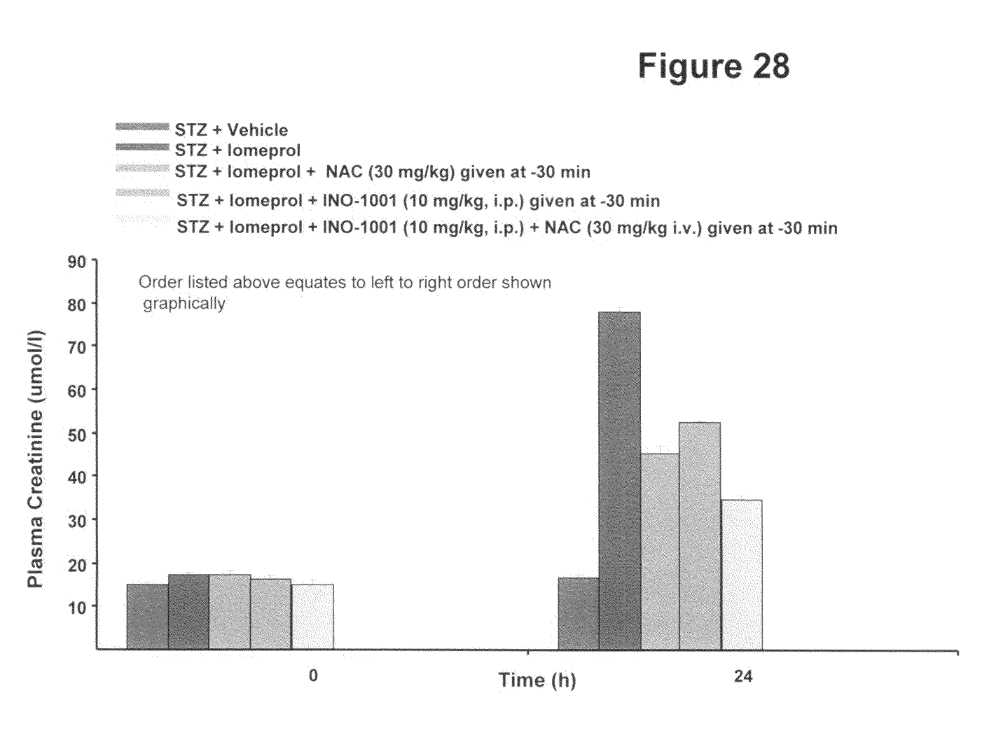

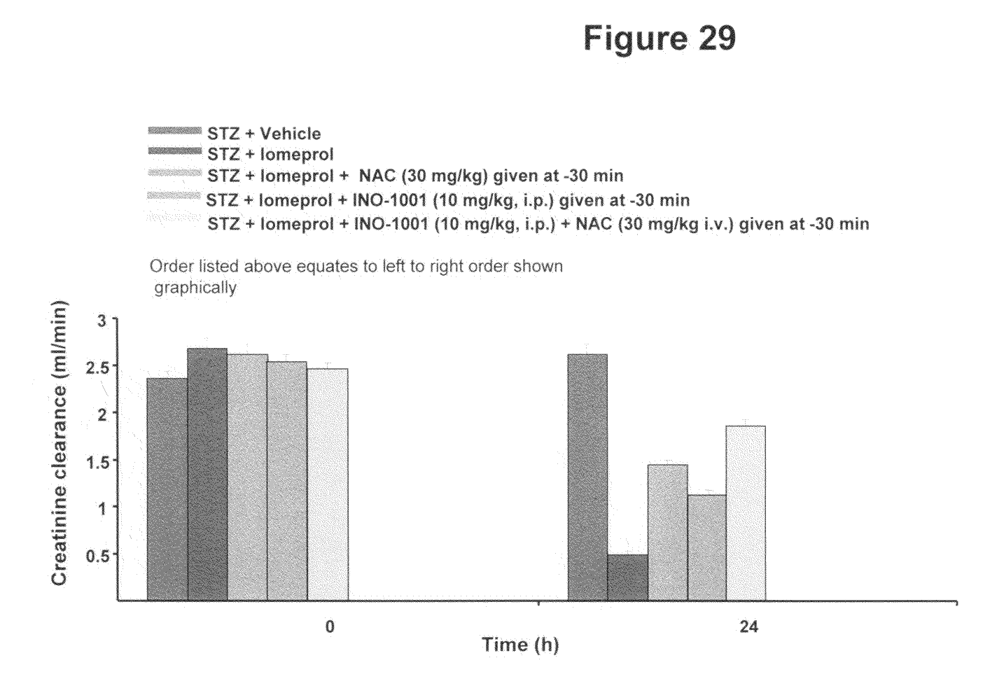

A significant increase of the plasma concentrations of creatinine (FIG. 28) as well as a significantly lower creatinine clearance (FIG. 29) was observed in diabetic rats at 24 h after the administration of the contrast agent. The treatment with NAC, INO-1001 as well as with NAC and INO-1001 produced significant reductions of plasma creatinine (FIG. 28) as well as a significantly increase in creatinine clearance (FIG. 29).

### Effect of INO-1001 and NAC on Plasma and Urine NGAL and Urine αGST Concentration.

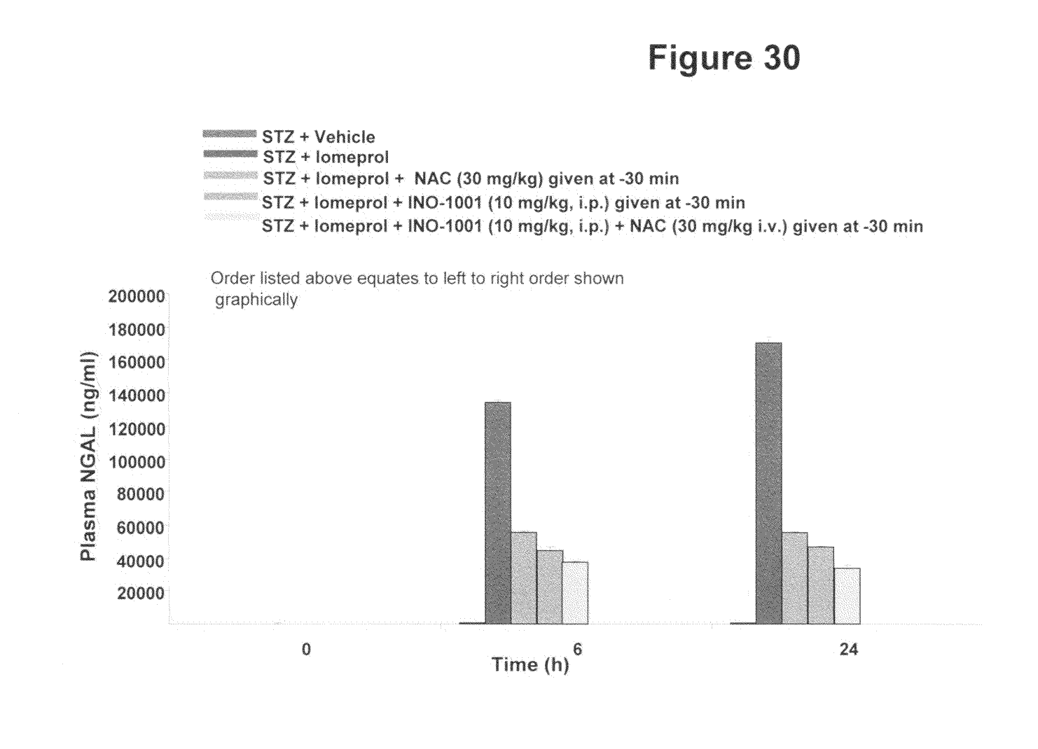

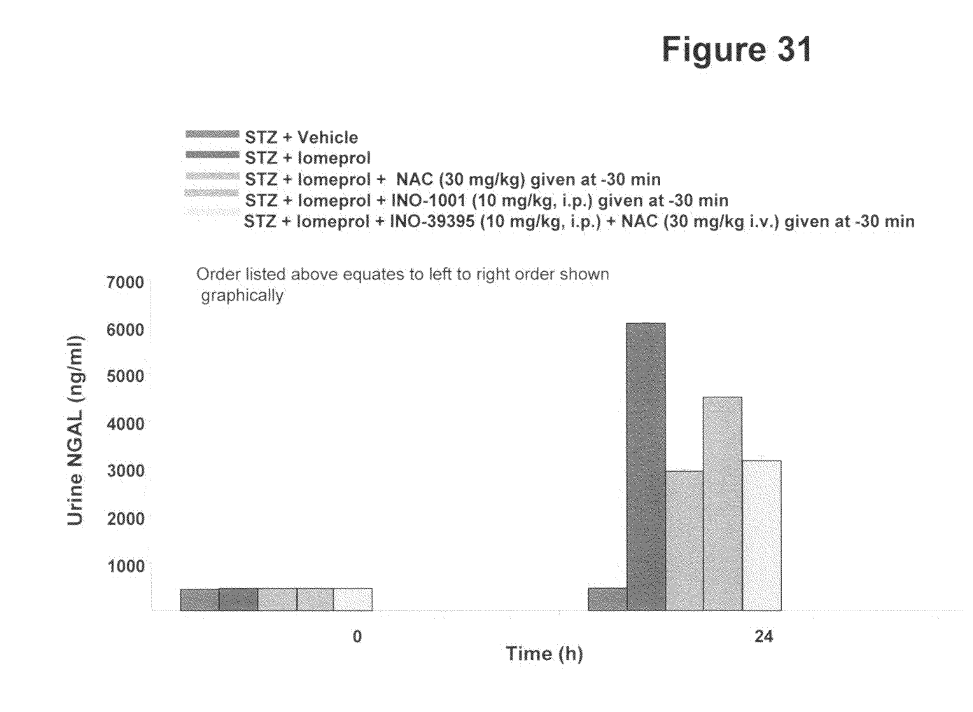

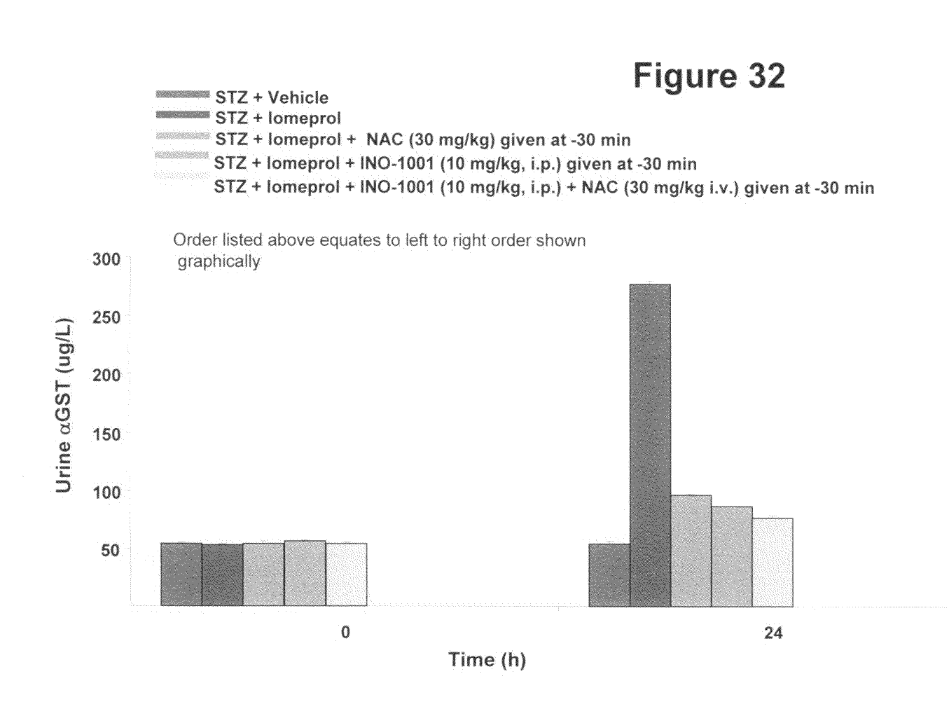

A significant increase of the plasma and urine concentrations of NGAL (FIGS. 30 and 31 respectively) and urine αGST (FIG. 32) was observed in diabetic rats at 24 hours after the administration of the contrast agent. The treatment with NAC, INO-1001 as well as with NAC and INO-1001 produced significant reductions of plasma and urine NGAL concentrations (FIGS. 30 and 31) and urine αGST (FIG. 32).

### Effect of INO-1001 and NAC on Urine Protein Concentration.

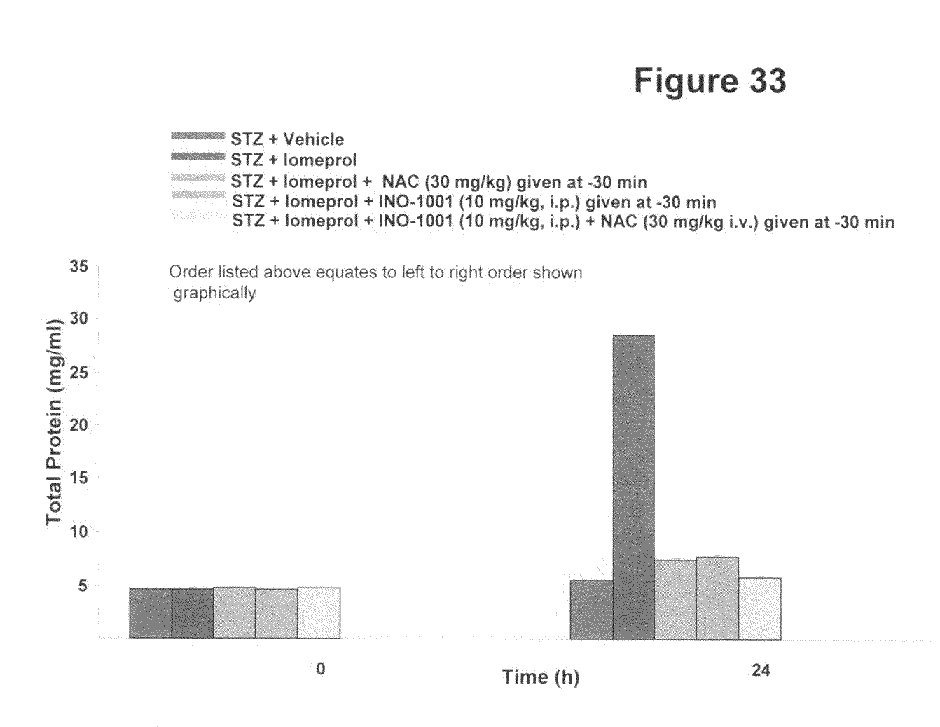

A significant increase of the urine concentrations of total protein (FIG. 33) was observed in diabetic rats at 24 hours after the administration of the contrast agent. The treatment with NAC, INO-1001 as well as with NAC and INO-1001 produced significant reductions of total protein concentration in the urine (FIG. 33).

### EQUIVALENTS

Those skilled in the art will recognize, or be able to ascertain using no more than routine experimentation, many equivalents of the specific embodiments of the invention described herein. Such equivalents are intended to be encompassed by the following claims. Any combination of the embodiments disclosed in the dependent claims is contemplated to be within the scope of the invention.

### INCORPORATION BY REFERENCE

All publications, patents, and pending patent applications referred to herein are hereby incorporated by reference in their entirety.

1. A method of preventing contrast-induced nephropathy including the step of administering an effective amount of a peroxynitrite decomposition agent to a subject to be administered a contrast agent.

2. The method as claimed in claim 1 wherein the peroxynitrite decomposition agent is administered to the subject prior to administration of a contrast agent.

3. The method as claimed in claim 1 wherein the peroxynitrite decomposition agent is administered to the subject simultaneously with the administration of the contrast agent.

4. The method as claimed in claim 1 wherein the peroxynitrite decomposition agent is administered to the subject after the administration of the contrast agent.

5. The method as claimed in claim 1 wherein the contrast agent is selected from Iothalamate, Metrizoate, Diatrizoate, Ioxilan Iohexyl, Ioversol, Iopamidol, Iopromide, Iomeprol, Ioxaglate, Iotrolan and Iodixanol.

6. The method as claimed in claim 5 wherein the contrast agent is selected from Iomeprol.

7. The method as claimed in claim 1 wherein the peroxynitrite decomposition agent is administered to the subject in an amount of between 1 ng/kg to 1000 mg/kg.

8. The method as claimed in claim 7 wherein the peroxynitrite decomposition agent is administered to the subject in an amount of between 0.01 mg to 100 mg.

9. The method as claimed in claim 8 wherein the peroxynitrite decomposition agent is administered to the subject in an amount of between 0.1 mg/kg to 10 mg/kg.

10. The method as claimed in claim 9 wherein the peroxynitrite decomposition agent is administered to the subject in an amount of between 0.1 mg/kg to 1 mg/kg.

11. The method as claimed in claim 1 wherein the peroxynitrite decomposition agent is a metalloporphyrin selected from a compound having the formula 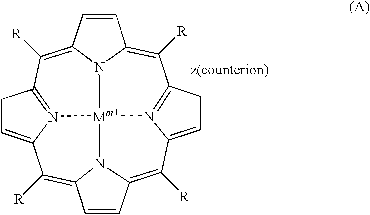
wherein:
M is Fe or Mn;
m is 0 or 1;
each R is independently selected from 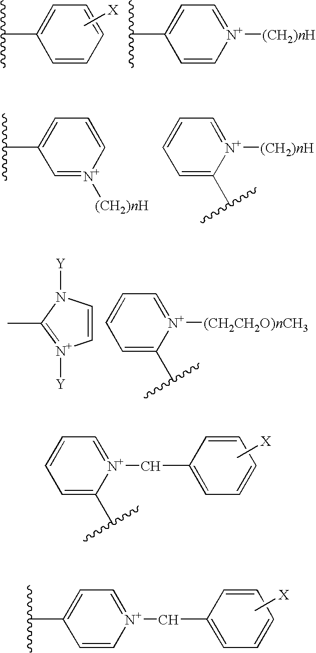
where X is selected from halogen, alkyl, —C(O)OH, —C(O)O⁻ or —C(O)(amino acid residue), SO₂OH, SO₂O⁻ or SO₂(amino acid residue);
where each Y is independently selected from halogen, C₁-C₆alkyl, C₁-C₆alkyl-O—C₁-C₆ alkyl,
each n is independently an integer from 1 to 4.
Z is the number of counterions sufficient to balance the charges of the compound of Formula (A).

12. The method as claimed in claim 11 wherein X is —C(O)(amino acid residue), the amino acid of the amino acid residue is β-alanine, γ-aminobutyric acid, 6-aminohexanoic acid, 5-aminovaleric acid, L-aspartic acid, L-glutamine, L-glutamic acid, glycine, L-phenylalanine, L-tyrosine, or L-valine.

13. The method as claimed in claim 11 wherein the counterion is Cl⁻ or Br⁻.

14. The method as claimed in claim 11 wherein the metalloporphyrin is selected from a compound having the formula 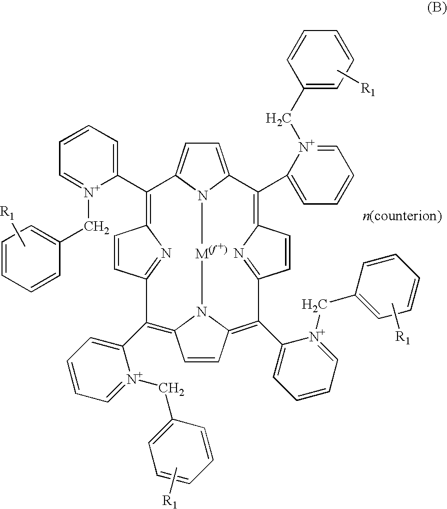
wherein:
M is Fe or Mn;
f is 0 or 1;
each R₁ is independently —C(O)OH, —C(O)O⁻ or —C(O)(amino acid residue) or SO₂(amino acid residue); and
n is the number of counterions sufficient to balance the charges of the compound of Formula (B).

15. The method as claimed in claim 14 wherein the counterion is Cl⁻ or Br⁻.

16. The method as claimed in claim 14 wherein the amino acid of the amino acid residue is β-alanine, γ-aminobutyric acid, 6-aminohexanoic acid, 5-aminovaleric acid, L-aspartic acid, L-glutamine, L-glutamic acid, glycine, L-phenylalanine, L-tyrosine, or L-valine.

17. The method as claimed in claim 16 wherein the amino acid residue is L-tyrosine.

18. The method as claimed in claim 11 or claim 14 wherein the metalloporphyrin is selected from

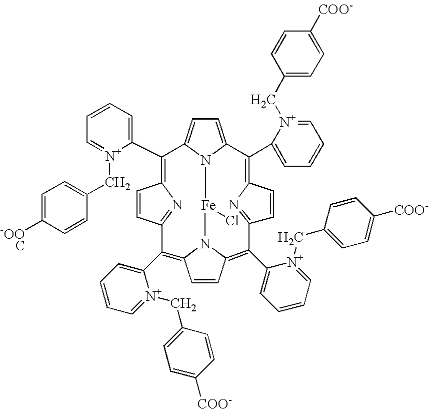

19. The method as claimed in claim 1 wherein the peroxynitrite decomposition agent is administered in combination with one or more of the following selection:
a prostaglandin; an adenosine antagonist, N-acetylcysteine (NAC), sodium bicarbonate, a calcium channel blocker, ascorbic acid, misoprostol, an ACE inhibitor, deferiprone, a PARP inhibitor, a superoxide dismutase (SOD) mimic, alpha-phenyl-N-tert-butyl nitrone, 2,4-disulphonyl-N-tert-butyl nitrone, 2-sulphonyl-N-tert-butyl nitrone, EPO and melatonin.

20. The method as claimed in claim 19 wherein the peroxynitrite decomposition agent is administered in combination with N-acetylcysteine (NAC).

21. A method of preventing contrast-induced nephropathy including the step of administering an effective amount of a superoxide dismutase mimic to a subject to be administered a contrast agent.

22. The method as claimed in claim 21 wherein the superoxide dismutase mimic is administered to the subject prior to administration of a contrast agent.

23. The method as claimed in claim 21 wherein the superoxide dismutase mimic is administered to the subject simultaneously with the administration of the contrast agent.

24. The method as claimed in claim 21 wherein the superoxide dismutase mimic is administered to the subject after the administration of the contrast agent.

25. The method as claimed in claim 21 wherein the contrast agent is selected from as Iothalamate, Metrizoate, Diatrizoate, Ioxilan Iohexyl, Ioversol, Iopamidol, Iopromide, Iomeprol, Ioxaglate, Iotrolan and Iodixanol.

26. The method as claimed in claim 25 wherein the contrast agent is selected from Iomeprol.

27. The method as claimed in claim 21 wherein the superoxide dismutase mimic is administered to the subject in an amount of between 1 ng/kg to 1000 mg/kg.

28. The method as claimed in claim 21 wherein the superoxide dismutase mimic is administered to the subject in an amount of between 0.01 mg/kg to 100 mg/kg.

29. The method as claimed in claim 21 wherein the superoxide dismutase mimic is administered to the subject in an amount of between 0.1 mg/kg to 10 mg/kg.

30. The method as claimed in claim 21 wherein the superoxide dismutase mimic is administered to the subject in an amount of between 0.1 mg/kg to 1 mg/kg.

31. The method as claimed in claim 21 wherein the superoxide dismutase mimic is selected from manganese tetrakis (4-benzoic acid) porphyrin, M40403, M40419, and AEOL 10113.

32. The method as claimed in claim 21 wherein the superoxide dismutase mimic is administered in combination with a peroxynitrite decomposition agent, N-acetylcysteine (NAC), sodium bicarbonate, a calcium channel blocker, ascorbic acid, prostaglandin E₁, misoprostol, an ACE inhibitor, deferiprone, a PARP inhibitor, alpha-phenyl-N-tert-butyl nitrone, 2,4-disulphonyl-N-tert-butyl nitrone, 2-sulphonyl-N-tert-butyl nitrone, a superoxide dismutase mimetic, an adenosine antagonist, EPO or melatonin.

33. The method as claimed in claim 32 wherein the superoxide dismutase mimic is administered in combination with N-acetylcysteine (NAC).

34. The method as claimed in claim 32 wherein superoxide dismutase mimic is administered in combination with a peroxynitrite decomposition agent selected from a metalloporphyrin compound having the formula 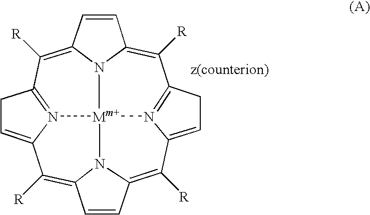
wherein:
M is Fe or Mn;
m is 0 or 1;
each R is independently selected from 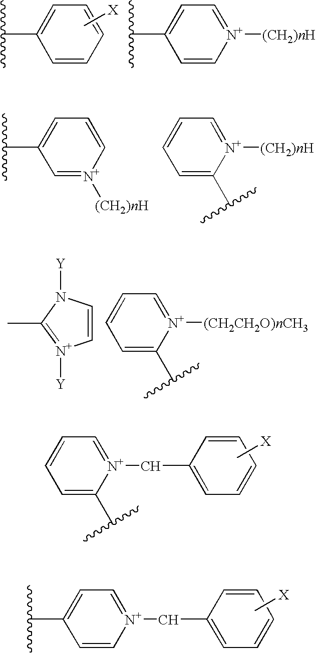
where X is selected from halogen, alkyl, —C(O)OH, —C(O)O⁻ or —C(O)(amino acid residue), SO₂OH, SO₂O⁻ or SO₂(amino acid residue);
where each Y is independently selected from halogen, C₁-C₆alkyl, C₁-C₆alkyl-O—C₁-C₆alkyl,
each n is independently an integer from 1 to 4,
Z is the number of counterions sufficient to balance the charges of the compound of Formula (A).

35. The method as claimed in claim 34 wherein X is —C(O)(amino acid residue) the amino acid of the amino acid residue is β-alanine, γ-aminobutyric acid, 6-aminohexanoic acid, 5-aminovaleric acid, L-aspartic acid, L-glutamine, L-glutamic acid, glycine, L-phenylalanine, L-tyrosine, or L-valine.

36. The method as claimed in claim 34 wherein the counterion is Cl⁻ or Br⁻.

37. The method as claimed in claim 34 wherein the metalloporphyrin is selected from a compound having the formula 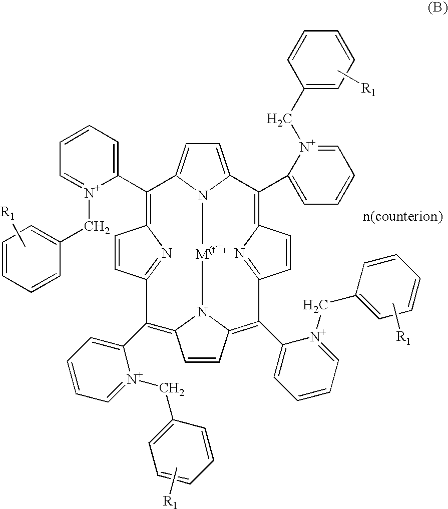
wherein:
M is Fe or Mn;
f is 0 or 1;
each R₁ is independently —C(O)OH, —C(O)O⁻ or —C(O)(amino acid residue) or SO₂(amino acid residue); and
n is the number of counterions sufficient to balance the charges of the compound of Formula (B).

38. The method as claimed in claim 37 wherein the counterion is Cl⁻ or Br⁻.

39. The method as claimed in claim 37 wherein the amino acid residue is β-alanine, γ-aminobutyric acid, 6-aminohexanoic acid, 5-aminovaleric acid, L-aspartic acid, L-glutamine, L-glutamic acid, glycine, L-phenylalanine, L-tyrosine, or L-valine.

40. The method as claimed in claim 39 wherein the amino acid residue is L-tyrosine.

41. The method as claimed in claim 37 wherein the metalloporphyrin is selected from

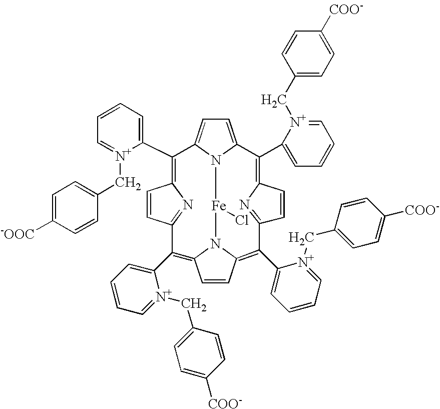

42. A method of preventing contrast-induced nephropathy including the step of administering an effective amount of a PARP inhibitor to a subject to be administered a contrast agent.

43. The method as claimed in claim 42 wherein the PARP inhibitor is administered to the subject prior to administration of a contrast agent.

44. The method as claimed in claim 42 wherein the PARP inhibitor is administered to the subject simultaneously with the administration of the contrast agent.

45. The method as claimed in claim 42 wherein the PARP inhibitor is administered to the subject after the administration of the contrast agent.

46. The method as claimed in claim 42 wherein the contrast agent is selected from as Iothalamate, Metrizoate, Diatrizoate, Ioxilan Iohexyl, Ioversol, Iopamidol, Iopromide, Iomeprol, Ioxaglate, Iotrolan and Iodixanol.

47. The method as claimed in claim 42 wherein the contrast agent is selected from Iomeprol.

48. The method as claimed in claim 42 wherein the PARP inhibitor is administered to the subject in an amount of between 1 ng/kg to 1000 mg/kg.

49. The method as claimed in claim 42 wherein the PARP inhibitor is administered to the subject in an amount of between 0.01 mg/kg to 100 mg/kg.

50. The method as claimed in claim 42 wherein the PARP inhibitor is administered to the subject in an amount of between 0.1 mg/kg to 10 mg/kg.

51. The method as claimed in claim 42 wherein the PARP inhibitor is administered to the subject in an amount of between 0.1 mg/kg to 1 mg/kg.

52. The method as claimed in claim 42 wherein the PARP inhibitor is selected from INO 1001, PJ34, ABT888, AG14699, AG14361, KU59346, BSI 201 and GPI 21016.

53. The method as claimed in claim 42 wherein the PARP inhibitor is administered in combination with a peroxynitrite decomposition agent, N-acetylcysteine (NAC), sodium bicarbonate, a calcium channel blocker, ascorbic acid, prostaglandin E₁, misoprostol, deferiprone, an ACE inhibitor, alpha-phenyl-N-tert-butyl nitrone, 2,4-disulphonyl-N-tert-butyl nitrone, 2-sulphonyl-N-tert-butyl nitrone, a superoxide dismutase mimetic, an adenosine antagonist, EPO or melatonin.

54. The method as claimed in claim 42 wherein the PARP inhibitor is administered in combination with N-acetylcysteine (NAC).

55. The method as claimed in claim 42 wherein the PARP inhibitor is administered in combination with a peroxynitrite decomposition agent.

56. The method as claimed in claim 55 wherein the peroxynitrite decomposition agent is a metalloporphyrin selected from a compound having the formula 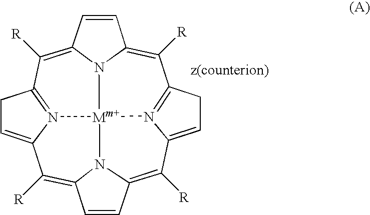
wherein:
M is Fe or Mn;
m is 0 or 1
each R is independently selected from 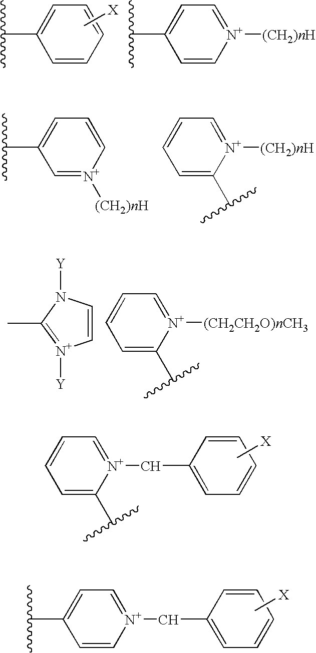
where X is selected from halogen, alkyl, —C(O)OH, —C(O)O⁻ or —C(O)(amino acid residue), SO₂OH, SO₂O— or SO₂(amino acid residue);
where each Y is independently selected from halogen, C₁-C₆alkyl, C₁-C₆alkyl-O—C₁-C₆alkyl,
each n is independently an integer from 1 to 4.
Z is the number of counterions sufficient to balance the charges of the compound of Formula (A).

57. The method as claimed in claim 56 wherein X is —C(O)(amino acid residue) the amino acid of the amino acid residue is β-alanine, γ-aminobutyric acid, 6-aminohexanoic acid, 5-aminovaleric acid, L-aspartic acid, L-glutamine, L-glutamic acid, glycine, L-phenylalanine, L-tyrosine, or L-valine.

58. The method as claimed in claim 56 wherein the counterion is Cl⁻ or Br⁻.

59. The method as claimed in claim 56 wherein the metalloporphyrin is selected from a compound having the formula 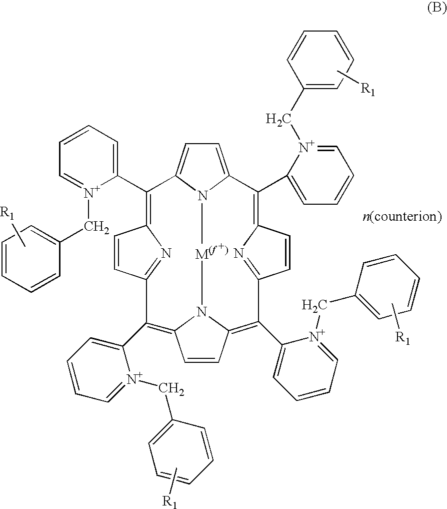
wherein:
M is Fe or Mn;
f is 0 or 1;
each R₁ is independently —C(O)OH, —C(O)O⁻ or —C(O)(amino acid residue) or SO₂(amino acid residue); and
n is the number of counterions sufficient to balance the charges of the compound of Formula (B).

60. The method as claimed in claim 59 wherein the counterion is Cl⁻ or Br⁻.

61. The method as claimed in claim 59 wherein the amino acid of the amino acid residue is β-alanine, γ-aminobutyric acid, 6-aminohexanoic acid, 5-aminovaleric acid, L-aspartic acid, L-glutamine, L-glutamic acid, glycine, L-phenylalanine, L-tyrosine, or L-valine.

62. The method as claimed in claim 61 wherein the amino acid residue is L-tyrosine.

63. The method as claimed in claim 59 wherein the metalloporphyrin is selected from [chemical structure]
